SEQUENCE LISTING (9 sequences)

SEQ ID NO: 1 - protein, 352 aa, Aspergillus oryzae
MRVTTLSTALFALTSTAVSAPTAGSSSPGLEVKLTQIDNTRVKAVVKNTGSEEVSFVHLNFFKDAGPVKKVSVYRGQDEVQFEGIKRRLRSSGITKEAVTSLGAGETLEDEFDIASTSDLASGGPVSIRSHGFVPIVVDGKITGYIPYKSNDLTVNVDGGKAAKVTKALSQLTRRTEVTDCKGDAESSLTTALSNAAKLANQAAEAAESGDESKFEEYFKTTDQQTRTTVAERLRAVAKEAGSTSGGSTTYHCNDPYGYCEPNVLAYTLPSKNEIANCDIYYSELPPLAQKCHAQDQATTTLHEFTHAPGVYQPGTEDLGYGYDAATQLSAQDALNNADSYALYANAIELKC

SEQ ID NO: 2 - protein, 402 aa, Aspergillus oryzae
MQSIKRTLLLLGAILPAVLGAPVQETRRAAEKLPGKYIVTFKPGIDEAKIQEHTTWATNIHQRSLERRGATGGDLPVGIERNYKINKFAAYAGSFDDATIEEIRKNEDVAYVEEDQIYYLDGLTTQKSAPWGLGSISHKGQQSTDYIYDTSAGEGTYAYVVDSGVNVDHEEFEGRASKAYNAAGGQHVDSIGHGTHVSGTIAGKTYGIAKKASILSVKVFQGESSSTSVILDGFNWAANDIVSKKRTSKAAINMSLGGGYSKAFNDAVENAFEQGVLSVVAAGNENSDAGQTSPASAPDAITVAAIQKSNNRASFSNFGKVVDVFAPGQDILSAWIGSSSATNTISGTSMATPHIVGLSLYLAALENLDGPAAVTKRIKELATKDWKDVKGSPNLLAYNGNA

SEQ ID NO: 3 - protein, 375 aa, Aspergillus oryzae
HASADAWASSKAISSTSSPSSFKMRFISVSSLLLALAPALNAVPVEVAGSAQGLDVTLSQVGNTRIKAWKNTGSEDVTFVHLNFFKDAAPVQKVSLFRNATEVQFQGIKQRLITEGLSDDALTTLAPGATIEDEFDIASTSDLSEGGTITINSNGLVPITTDNKVTGYIPFTSNELSIDVDAAEAASVTQAVKILERRTKVTSCSGSRLSALQTALRNTVSLARAAATAAQSGSSSRFQEYFKTTSSSTRSTVAARLNAVANEAASTSSGSTTYYCSDVYGYCSSNVLAYTLPSYNIIANCDLYYSYLPALTSTCHAQDQATTTLHEFTHAPGVYSPGTDDLGYGYSAATALSASQALLNADTYALFANAVNLNC

SEQ ID NO: 4 - protein, 9 aa, Aspergillus oryzae
TEVTDCKGD

SEQ ID NO: 5 - protein, 9 aa, Aspergillus oryzae
GLTTQKSAP

SEQ ID NO: 6 - protein, 10 aa, Aspergillus oryzae
TKVTSNSGSR

SEQ ID NO: 7 - protein, 12 aa, Aspergillus oryzae
ARGVALARGARG

SEQ ID NO: 8 - protein, 9 aa, Aspergillus oryzae
HEEFEGRAS

SEQ ID NO: 9 - protein, 9 aa, Aspergillus oryzae
KRIKELATK

(19) United States
(12) Patent Application Publication
(10) Pub. No.: US 2008/0081035 A1
(43) Pub. Date: Apr. 3, 2008

(54) Therapeutic protease compositions

(75) Inventors: Michael J. Parmely, Kansas City, MO (US); Rohit Medhekar, Springfield, MO (US); Anthony Collier, Forsyth, MO (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: National Enzyme Company, Forsyth, MO (US)

(21) Appl. No.: 11/541,647

(22) Filed: Oct. 3, 2006

(51) Int. Cl. A61K 38/48

(52) U.S. Cl. 424/94.63

(57) ABSTRACT

A method of treating an inflammatory condition involving TNF-α in a mammal by administering to a patient a composition with an effective amount of an isolated alkaline protease in an amount effective to inactive TNF-α. The invention also involves compositions, including pharmaceutical compositions containing an isolated alkaline protease in an amount effective to inactive TNF-α especially those from Aspergillus oryzae.

### FIELD OF THE INVENTION

The present invention relates generally to a method of treating mammalian disease utilizing protelotyic enzymes of plant, animal, and/or microbial origin. In particular, this invention relates to the therapeutic value of an alkaline protease isolated from the filamentous fungus Aspergillus oryzae for the treatment of inflammatory disorders mediated by TNF-α.

### BACKGROUND OF THE INVENTION

Inflammatory bowel disease (IBD) is a collection of chronic disorders that include Crohn's disease, ulcerative colitis, and celiac disease. The incidence of these diseases has been increasing in developed countries over the past four decades. Recent advances in our understanding of the immunopathogenic mechanisms underlying these conditions have afforded new therapeutic approaches that target specific components of the inflammatory process. Cytokines, including tumor necrosis factor-α (TNF-α), are now known to play central roles in many forms of IBD as evidenced by the efficacy of anti-TNF-α drugs in their treatment. However, these therapies have a number of shortcomings, not the least of which is their costs.

Crohn's disease (CD) has an estimated incidence in North America approaching 200 cases/100,000 per year, a rate that has increased 8-10-fold since the 1960s. The prevalence of the disease is approximately 1 in 500 Americans. Despite drug therapy, up to 70% of CD patients undergo corrective surgery, and relapse after conventional therapies (corticosteroids, azathioprine, or methotrexate) is common. Recent advances in the use of biological therapies (e.g., anti-TNF-α monoclonal antibody; INFLIXIMAB) have dramatically improved outcomes. However, the cost of treating a single CD patient with Infliximab ranges from $25,000 to $30,000 per year, excluding the cost of clinic visits necessary for intravenous injection of the drug. In addition, the systemic delivery of anti-TNF-α biologicals has been associated with an increased susceptibility to mycobacterial pneumonia, indicating the important role the cytokine plays in protective immune responses to intracellular microbial pathogens. Azathioprine and corticosteroids have similar side effects due to their nonspecific immunosuppressive activities. Adverse allergic reactions to Infliximab (infusion reactions) have also been reported.

CD has a clear immunological component initiated by innate immune responses to microbial flora. The chronic nature of CD is maintained by the persistent activation of inflammatory cells, including Th1 lymphocytes and submucosal macrophages. The cytokines interleukin-12 (IL-12), interferon-γ (IFN-γ), and tumor necrosis factor-α (TNF-α) play central roles in disease pathogenesis, and affected intestinal tissues of CD patients show marked elevations in the levels of TNF-α mRNA. Inducing the apoptosis of Th1 cells with the immunosuppressive agent azathioprine or neutralizing the activity of TNF-α with the monoclonal antibody Infliximab are both effective therapies for this disease in human beings. Similar cytokine-driven chronic inflammation characterizes ulcerative colitis, although the key mediators are not as clearly defined. In addition, proteases derived from microorganisms such as Aspergillus oryzae modify the course of inflammation and other body processes by selectively interacting with pro-inflammatory cytokines.

Proteolytic enzymes have a number of commercial applications and constitute one of the largest industrial classes of enzymatic proteins. Commercially important proteases are derived from plant, animal, and microbial sources and are available either as semi-purified or recombinant preparations. Proteases derived from Bacillus and Aspergillus species are among the most frequently used microbe-derived products and are often supplied as mixtures of several different enzymes. As such, these formulations can be active over a wide pH range and can show broad substrate specificities. Among the most common industrial and commercial applications of microbial proteases are their use in detergents, leather processing, and food production (cheese production, wheat gluten digestion, soy sauce production, debittering of food components, and aspartame production). For example, the alkaline protease of Aspergillus oryzae is an effective flavor-enhancing agent in the manufacture of soy sauce.

Proteolytic enzymes from animal or plant sources, such as trypsin, chymotrypsin, pepsin, papain, and bromelain, have utility as digestive enzymes.

Proteolytic enzymes also have been used in anti-inflammatory compositions. For example, proteolytic enzymes such as bromelain, papain, trypsin, and chymotrypsin have been traditionally used as anti-inflammatory agents, usually in the form of buccal tablets. In particular, microbial protease formulations such as Wobenzym N; Phlogenzym; Mulsal; and Wobe-Mugos E have been used as anti-inflammatories. Zhou et al. (1983) describes a method of intraduodenal injection of neutral peptidase isolated from Bacillus subtilis which showed a strong anti-inflammatory effect with low toxicity in a rat model.

Further, proteases from Aspergillus oryzae have been used in therapeutic methods as disclosed in U.S. Pat. No. 6,413,512 (Jul. 2, 2002) Houston et al., which describes crude preparations containing a mixture of Aspergillus oryzae proteases. Thus proteases, especially from Aspergillus oryzae, are not pathogenic in humans and are safe for the treatment of inflammatory conditions including gastrointestinal diseases. However, mixtures of proteases that are non-specific can affect patients broadly and a more specific therapy and more pure preparations of proteases are needed.

### BRIEF SUMMARY OF THE INVENTION

It is an object of the present invention to provide a method of treating and/or prevention of inflammation which involves TNF-α in mammals. Another object of the present invention is to provide a method of treating and/or prevention of the recurrence of mammalian inflammatory diseases which involve TNF-α. Yet another object of the present invention is to provide a method of treating and/or prevention of the symptoms of mammalian inflammatory disease which involve TNF-α. Specifically, the object of the invention is the treatment of inflammatory disorders mediated by TNF-α.

These and other objects of the invention are met by one or more of the following embodiments.

In one embodiment, this invention provides a method of treating anti-inflammatory condition involving TNF-α in a mammal comprising administering to said mammal a composition comprising an isolated alkaline protease in an amount effective to inactivate TNF-α. The inflammatory conditions suitable for treatment by administration of the composition comprising alkaline protease include but are not limited to diseases, conditions, maladies, illnesses that are related to, mediated by, caused by, exacerbated, aggravated, or the symptoms worsened by TNF-α. In particular, inflammatory conditions suitable for treatment by administration of the composition comprising alkaline protease include but are not limited to ulcerative colitis, asthma, Parkinson's disease (PD), cardiovascular disease, Crohn's disease (CD), multiple sclerosis (MS), irritable bowel syndrome (IBS), inflammatory bowel disease (IBD), irritable bowel disease, Alzheimer's disease (AD), infection, soft tissue injury, encephalitis, Graves' disease, myasthenia gravis, rheumatoid arthritis (RA), scleroderma, acute rheumatic fever, Kawasaki disease (KD), cachexia syndrome, acute pancreatitis, and chronic heart failure (CHF).

In another embodiment, the invention is directed to a use of a composition comprising an isolated alkaline protease for medical therapy. The medical therapy according to this invention is preferred to be for inflammatory conditions, disorders, and diseases, most preferred wherein said conditions, disorders, and diseases involved TNF-α. In particular, inflammatory conditions suitable for treatment by administration of the composition comprising alkaline protease include but are not limited to ulcerative colitis, inflammatory bowel disease (IBD), asthma, cardiovascular disease, Crohn's disease (CD), multiple sclerosis (MS), irritable bowel syndrome (IBS), Alzheimer's disease (AD), Parkinson's disease (PD), infection, soft tissue injury, encephalitis, Graves' disease, myasthenia gravis, rheumatoid arthritis (RA), scleroderma, acute rheumatic fever, Kawasaki disease (KD), cachexia syndrome, acute pancreatitis, and chronic heart failure (CHF).

Preferably, the composition comprising alkaline protease does not include a 26 kDa protease or deuterolysin. Also, the comprising alkaline protease can consist essentially of an isolated alkaline protease in an amount effective to inactive TNF-α. Typically the composition will have an optimum proteolytic activity at about pH=8.0. Preferably, the composition will have a maximum proteolytic activity in the range of from about pH 6.0 to 10.0.

Preferably, the isolated alkaline protease in the composition will be an Aspergillus oryzae alkaline protease, more preferably the isolated Aspergillus oryzae alkaline protease will comprising SEQ ID NO: 2. The isolated Aspergillus oryzae (A. oryzae) alkaline protease of the composition can be recombinantly produced in microbial, plant, insect, mammalian cells or mammalian hosts. The recombinantly produced alkaline protease can be made as a fusion protein, preferably with a cleavable linkage, and most preferably as a secreted fusion protein with a cleavable linkage.

In one embodiment, the composition comprising alkaline protease effective to treat inflammatory conditions is administered orally, injected, inhaled, or via suppository. Preferably, the composition is administered orally or buccally. Further said composition can comprise a pharmaceutically acceptable carrier, excipient, diluent, or solution. And, said composition can be a food supplement, a nutritional supplement, or a food product. The compositions suitable for use in the instant invention may comprise alkaline protease, consisting of alkaline protease, and/or consist essentially of alkaline protease (i.e., composition suitable for this invention may contain alkaline protease as the only active ingredient for anti-inflammatory treatment, wherein the balance of the composition is non-active ingredients—e.g., carriers, excipients, diluents, fillers).

The prior art provided mixtures of proteases that are non-specific which can affect patients broadly. Surprisingly, the present inventors have discovered a more specific therapy using more pure preparations of protease which retain the efficacy against TNF-α shown by prior art mixtures, but have less activity against other physiological components.

### BRIEF DESCRIPTION OF THE DRAWINGS

FIG. 1

A diagram of a production process to produce a protease powder containing alkaline protease.

FIG. 2

Source 30Q chromatography of a protease powder containing alkaline protease. The protease powder containing alkaline protease as prepared by Example 1 was loaded onto Source 30Q in 30 mM Tris, pH 8.0 and the unbound peak was collected. This peak had proteolytic activity (shaded area) and was designated Peak I. The remaining proteins were eluted with a linear 0-500 mM NaCl gradient in 30 mM Tris, pH 8.0. Peak IV also had protease activity when measured against protamine (shaded area).

FIG. 3

Source 30S Chromatography of Peak I from Source 30Q. 30Q Peak I was applied to Source 30S exchanger in 10 mM sodium acetate buffer, pH 5.5, and bound proteins were eluted with a linear NaCl gradient (0-250 mM) in the same buffer. Both eluted peaks had protease activity against the substrate protamine (shaded area) and were designated 30S Peak I and 30S Peak II.

FIG. 4

SDS-PAGE of purified A. oryzae proteases. Shown here are sample of protease powder containing alkaline protease as prepared by Example 1 (“Prep.”; lane 1) and three active protease peaks from ion exchange chromatography: 30Q Peak IV (lane 2), 30S Peak II (lane 3), and 30S Peak I (lane 4). The proteins were electrophoresed under reducing conditions on a 15% polyacrylamide SDS gel and visualized after staining with Coomassie Brilliant Blue. The major protein bands identified by squares had apparent molecular masses of 30 kDa (lane 2), 36 kDa (upper band of lane 3), 28 kDa (lower band of lane 3) and 26 kDa (lane 4). The gel was then blotted onto a PVDF membrane, and the indicated bands (dashed squares) were sequenced. Results of N-terminal amino acid sequencing are shown in Table 2. The letters below each lane refer to the ultimately determined identities of the proteins (Table 2).

FIG. 5

Amino acid sequence of Deuterolysin (SEQ ID NO: 1).

FIG. 6

Amino acid sequence of Alkaline Protease (SEQ ID NO: 2).

FIG. 7

Amino acid sequence of 26 kDa Protease (SEQ ID NO: 3).

FIG. 8

A scheme for the isolation of Aspergillus oryzae proteases.

FIG. 9A-B

Elution profile for Source 30Q (Peaks I and IV) (A) and Elution profile from Source 30S (Peak I and II) (B).

FIG. 10A-D

Effects of the purified A. oryzae proteases on human recombinant TNF-α: (A) protease powder prepared according to Example 1 (preparation); (B) alkaline protease; (C) 26 kDa protease; and (D) deuterolysin. 50 ng quantities of TNF-α were incubated at 37° C. for 18 hours with the indicated quantities of protease powder prepared according to Example 1, alkaline protease, 26 kDa protease or deuterolysin. Western blotting was then used to determine substrate specificity. C=control, buffer-treated TNF-α.

FIG. 11A-D

Effects of purified A. oryzae proteases on the bioactivity of human TNF-α: (A) protease powder prepared according to Example 1 (preparation); (B) alkaline protease; (C) 26 kDa protease; and (D) deuterolysin. 50 ng quantities of TNF-α were incubated at 37° C. for 24 hours with the indicated quantities (∘=buffer alone; =5 ng; ▪=10 ng) of: protease powder prepared according to Example 1 (preparation); alkaline protease; 26 kDa protease; and deuterolysin. The reactions were then stopped by addition of FBS and samples were added to C2C12 cells that had been costimulated with mouse IFN-γ. Twenty-four hours later, nitrite was measured in culture fluids.

FIG. 12A-D

Effects of the purified A. oryzae proteases on human recombinant IFN-γ: (A) protease powder prepared according to Example 1 (preparation); (B) alkaline protease; (C) deuterolysin; and (D) 26 kDa protease. 50 ng quantities of human IFN-γ were incubated at 37° C. for 18 hours with the indicated quantities of preparation, alkaline protease, 26 kDa protease or deuterolysin. Western blotting was then used to determine substrate specificity. C=control, buffer-treated IFN-γ.

FIG. 13A-B

Effects of the purified A. oryzae proteases on the Fc receptor-inducing activity of human IFN-γ. Samples of the cytokine were first treated with the indicated Pseudomonas or fungal proteases: Elastase (∘); Preparation (); Alkaline Protease (□); Deuterolysin (▪); and 26 kDa Protease (AΔ. The treated samples were added to human promyelocytic U937 cells to induce expression of Fc receptors for IgG, which was detected by flow cytometry. The results are plotted either as a decrease in of mean channel fluorescence (A) or a decrease in % positive cells (B) plotted against increasing protease amounts.

FIG. 14A-C

Degradation of mouse recombinant TNF-α (A) or IFN-γ (B) following treatment with: the protease powder prepared according to Example 1 (preparation), purified alkaline protease (AP); Pseudomonas elastase (E); and buffered-treated control (C). The cytokines (100 ng each) were incubated at 37° C. overnight with the indicated quantities (ng) of enzymes, and then analyzed by Western blotting for evidence of degradation. A scan of AP-treated mouse TNF-α (left-hand Western blot) showing the decreasing density of the top band as a function of alkaline protease concentration (C).

FIG. 15A-D

Effects of three purified A. oryzae proteases on the bioactivity of mouse TNF-α: (A) protease powder prepared according to Example 1 (preparation); (B) alkaline protease; (C) 26 kDa protease; and (D) deuterolysin. 50 ng quantities of TNF-α were incubated at 37° C. for 24 hours with the indicated quantities of the protease powder prepared according to Example 1 or one of the three purified proteases. The reactions were then stopped by addition of FBS and samples were added to C2C12 cells that had been costimulated with mouse IFN-γ. Twenty-four hours later, nitrite was measured in culture fluids.

FIG. 16A-B

The protease powder prepared according to Example 1 inactivates mouse recombinant TNF-α. 50 ng of the cytokine was treated for 24 hours with either 0 (□); 5 (∘); or 10 ng () of either Pseudomonas elastase (A) or protease powder prepared according to Example 1 (B). Then the residual bioactivity of the cytokine was measured by its ability to co-activate mouse C2C12 myoblast cells in the presence of excess IFN-γ for the production of NO (nitrite). Treatment of the mouse TNF-α with either protease led to a loss of its biological activity.

FIG. 17A-D

Effects of three purified A. oryzae proteases on the bioactivity of mouse IFN-γ: (A) protease powder prepared according to Example 1 (preparation); (B) alkaline protease; (C) 26 kDa protease; and (D) deuterolysin. 50 ng quantities of mouse recombinant IFN-γ were incubated for 24 hours at 37° C. with either buffer or 2.5-10 ng of the indicated protease: (∘) Buffer; (▴) 2.5 ng; () 5 ng; (▪) 10 ng. IFN-γ bioactivity was determined as coinduction of NO production by C2C12 cells.

FIG. 18

pH Profile of Protease Powder Prepared According to Example 1.

FIG. 19A-C

TNF-α was first treated either with PBS, AP or D for 2 h, the reaction mixtures were inactivated by the addition of FBS, and the cytokine-protease mixtures were injected i.p. into mice. (A) One hour later the mice were euthanized, samples of their intestines were collected and fixed, and tissue sections were analyzed by immunohistology for the expression of activated caspase-3. These are the results are shown in which data are expressed in two forms: (B) the frequency distributions of sections expressing different levels of activated caspase-3; and (C) the percentages of tissue sections in which 10% or more of the villi stained for activated caspase-3. Treating TNF-α with increasing concentrations of AP, but not D, destroyed the ability of the cytokine to activate caspase-3 in intestinal epithelial cells.

FIG. 20A-B

In this figure, various amounts of deutrolysin and alkaline protease were administered to mice 15 min prior to challenge with TNF-α. Intestinal tissues were again recovered 1 h later and the expression of activated caspase-3 was measured. (A) Pretreating mice with AP at doses of 1-10 μg blocked the action of TNF-α in vivo, whereas pretreatment with D was without a significant inhibitory effect. (B) The ability of AP to provide protection against the apoptotic effects of TNF-α in vivo was limited in its duration. Thus, pretreatment of the mice for up to 30 min prior to TNF-α challenge prevented caspase-3 activation, whereas delaying cytokine challenge beyond 30 min diminished the protective effect of the protease.

### DETAILED DESCRIPTION OF THE INVENTION

### Definitions

Unless otherwise indicated, all terms used herein have the same meaning as they would to one skilled in the art.

The term “alkaline protease”, as used in this specification and claims, indicates a protease or protease mixture, which has maximum proteolytic activity in the range of from about pH 6-10, most preferably of about pH˜8. The alkaline protease has maximum proteolytic activity wherein it is shows at least 80% of its maximal activity level from about pH 6-10. Preferably, the alkaline protease has a maximum activity at about pH 7-9. Also, the alkaline protease only shows one major activity peak between pH 6-10 and no peaks at pH 4-6.

It must be noted that, as used in this specification and the appended claims, the singular forms “a,” “an,” and “the” include plural referents unless the context clearly dictates otherwise. Thus, for example, reference to “an antioxidant” includes a mixture of one or more antioxidants, reference to “a vitamin” includes reference to one or more of such vitamins, and reference to “a microbial protease” includes references to one or more of such microbial proteases.

The Aspergillus species according to the invention are preferably selected from the group of Aspergillus aculeatus, Aspergillus auricomus, Aspergillus caesillus, Aspergillus conicus, Aspergillus ficuum, Aspergillus flavus, Aspergillus foetidus, Aspergillus fumigatus, Aspergillus japonicus, Aspergillus nidulans, Aspergillus niger, Aspergillus nomius, Aspergillus ochraceus, Aspergillus oryzae, Aspergillus ostianus, Aspergillus parasiticus, Aspergillus parasiticus, Aspergillus phoenicus, Aspergillus phoenicus, Aspergillus restrictus, Aspergillus sake, Aspergillus soja (Aspergillus sojae), Aspergillus sydowii, Aspergillus tamari, Aspergillus terreus, Aspergillus ustus, and Aspergillus versicolor. The most preferred species for use in the instant invention is Aspergillus oryzae (A. oryzae).

“Therapy” or “therapeutic” as used herein, refers broadly to treating a disease, arresting or reducing the development of the disease or its clinical symptoms, and/or relieving the disease, causing regression of the disease or its clinical symptoms. Therapy encompasses prophylaxis, prevention, treatment, cure, regimen, remedy, minimization, reduction, alleviation, and/or providing relief from a disease, signs, and/or symptoms of a disease. Therapy encompasses an alleviation of signs and/or symptoms in patients with ongoing disease signs and/or symptoms, e.g. of inflammation. Therapy also encompasses “prophylaxis” and “prevention”. Prophylaxis includes preventing disease occurring subsequent to treatment of a disease in a patient or reducing the incidence or severity of the disease in a patient. The term “reduced”, for purpose of therapy, refers broadly to the clinical significant reduction in signs and/or symptoms. Therapy includes treating relapses or recurrent signs and/or symptoms, e.g. of inflammation. Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. Therapy can be for patients with risk factors, at risk patients in a susceptible population, patients with a history of disease, patients with symptoms, patients with signs, patients with signs but no symptoms, and patients with symptoms but no signs. Therapy can also be for patients without risk factors, not at risk, patients not in a susceptible population, patients no history of disease, patients with no symptoms, patients without signs. Therapy can alleviate, allay, abate, assuage, curtail, decrease, ease, lessen, lighten, make better, make healthy, mitigate, mollify, pacify, relieve, rehabilitate, remedy, repair, and/or soothe a disease, disease signs, and/or disease symptoms.

“Treating” or “treatment”, as used herein, refers broadly to a course of therapy where signs and/or symptoms are present in the patient. The term “reduced”, for purpose of therapy, refers broadly to clinically significant reduction in signs and/or symptoms. Treatment includes treating chronic disease (“maintenance”) and acute disease. Treatment can be for patients with risk factors, at risk patients in a susceptible population, patients with a history of disease, and/or patients with symptoms, patients with signs. Treatment can alleviate, allay, abate, assuage, curtail, decrease, ease, lessen, lighten, make better, make healthy, mitigate, mollify, pacify, relieve, rehabilitate, remedy, repair, and/or soothe a disease, disease signs, and/or disease symptoms.

“Prophylaxis”, as used herein, refers broadly to a course of therapy where signs and/or symptoms are not present in the patient, are in remission, or were previously present in a patient. The term “reduced”, for purpose of therapy, refers broadly to the clinical significant reduction in the incidence of signs and/or symptoms. Prophylaxis includes preventing disease occurring subsequent to treatment of a disease in a patient or reducing the incidence or severity of the disease in a patient. Prophylaxis encompasses “prevention” and as used herein, is a subset under “therapy” and refers broadly to inhibiting the disease, arresting the development of the disease or its clinical symptoms, and/or relieving the disease, causing regression of the disease or its clinical symptoms. Prevention also preferably includes prophylaxis as preventing or reducing incidence or severity of disease in a patient. Further, prevention includes treating patients who can potentially develop the disease, especially patients who are susceptible to the disease, e.g. members of a patent population, those with risk factors, or at risk for developing the disease. Prevention also includes preventing relapses or the recurrence of signs and/or symptoms, e.g. inflammation.

“Signs” of disease, as used herein, refers broadly to any abnormality indicative of disease, discoverable on examination of the patient; an objective indication of disease, in contrast to a symptom, which is a subjective indication of disease.

“Symptoms” of disease as used herein, refers broadly to any morbid phenomenon or departure from the normal in structure, function, or sensation, experienced by the patient and indicative of disease.

“Therapeutically effective amount” as used herein, refers broadly to the amount of a compound that, when administered to a patient for treating a disease, is sufficient to effect such treatment for the disease. The therapeutically effective amount can be an amount effective for prophylaxis, and/or an amount effective for prevention. The therapeutically effective amount can be an amount effective to reduce inflammation, an amount effective to prevent inflammation, to reduce the severity of inflammation, to eliminate inflammation, to slow the development of inflammation, to prevent the development of inflammation, and/or effect prophylaxis of inflammation. The “therapeutically effective amount” can vary depending on the disease and its severity and the age, weight, medical history, predisposition to conditions, preexisting conditions, etc., of the patient to be treated. The term “effective amount” is taken to be synonymous with “therapeutically effective amount” for purposes of this invention.

“Prophylatically effective amount” as used herein, refers broadly to the amount of a compound that, when administered to a patient for prophylaxis or prevention of a disease or reoccurrence of a disease, is sufficient to effect such prophylaxis or prevention for the disease or reoccurrence. The prophylatically effective amount is, in particular, an amount effective for prophylaxis, and/or an amount effective for prevention. The prophylatically effective amount can be an amount effective to prevent inflammation, to reduce the severity of inflammation if it reoccurs, to slow the development of inflammation, to prevent the development of inflammation, and/or effect prophylaxis of inflammation. The “prophylatically effective amount” can vary depending on the disease and its severity and the age, weight, medical history, predisposition to conditions, preexisting conditions, etc., of the patient to be treated. The term “effective amount” is taken to be synonymous with “prophylatically effective amount” for purposes of this invention.

“Administration” as used herein, refers broadly to any means by which a composition is given to a patient.

“Patient” as used herein, refers broadly to any animal who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient can be a clinical patient such as a human or a veterinary patient such as a companion, domesticated, livestock, exotic, or zoo animal. Animals can be mammals, reptiles, birds, amphibians, or invertebrates.

“Mammal” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to humans, non-human primates, alpacas, capybaras, cats, chimpanzees, chinchillas, cows, dogs, goats, gorillas, horses, llamas, mice, pigs, rats, sheep, and tapirs. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C., which is incorporated by reference. Effects of protease mixtures

Proteases can modify the course of inflammation and other normal or disease processes via selective inactivation by means of proteolysis of protein and peptide mediators of disease or normal body functions. Proteases cleave biologically relevant proteins and peptides destroying their biological activities in a selective fashion that reflects both the primary, secondary and tertiary structure of these proteins and peptides and the specificity of the proteases.

The specificity of the proteases can be demonstrated by comparing two or more proteases with regard to their ability to inactivate a given biologically active protein. The specificity of the proteases can be demonstrated by comparing the effect of a given protease on two or more biologically active proteins (substrates).

In general, proteases can be separated from one another and, when separated, show unique properties, including distinct specificities for biologically active proteins and peptides. For instance, individual proteases purified from Aspergillus oryzae (A. oryzae) show unique specificity for biologically active proteins. This property of a protease's specificity affords the opportunity to limit adverse effects by administering a purified reagent with a limited range of activities in vivo. The corollary is that a mixture limited to one or a few proteases may have fewer or no serious side effects.

This property affords the opportunity to combine purified proteases in various ratios to create compositions with identifiable specificities under selected application conditions. This property affords the opportunity to enhance the activity of the proteases as it is well-recognized that proteases can act synergistically on protein and peptide substrates. This property endows mixtures of proteases with wider ranges of specificity and stability in vivo, if that is a desirable characteristic for a particular application.

### Therapeutic Utilities of Alkaline Protease

In inflammatory conditions, chemokines attract inflammatory cells to the lungs and promote pulmonary damage. Orally administered proteases are effective at limiting the action of these peptide chemokines. The therapeutic utility of the alkaline protease is based on observations in vitro and in a mouse model that Aspergillus oryzae (A. oryzae) alkaline protease selectively cleaves and inactivates a known mediator of inflammation, tumor necrosis factor-α (TNF-α). Alkaline protease administered as either a mixture or a purified protease cleaves and inactivates TNF-α. The alkaline protease described herein may be used in a method of treating an inflammatory condition mediated by TNF-α in a mammal comprising administering to said mammal a composition comprising an effective amount of an isolated alkaline protease in an amount effective to inactive TNF-α by means of cleavage.

For purposes of this invention, mouse models of inflammatory bowel disease (IBD) are suitable experimental vehicles for testing the effectiveness of alkaline protease on mucosal inflammatory diseases. Using an animal model of acute TNF-α-induced intestinal inflammation, the inventors have shown that alkaline protease has significant therapeutic use as an anti-TNF-α reagent. Aspergillus oryzae (A. oryzae) alkaline protease provides substantial value in the prevention of TNF-α-dependent inflammatory lesions in the gut.

The bioactivity of mouse TNF-α can be decreased in vivo by prior treatment of mice with Aspergillus oryzae proteases. Aspergillus oryzae alkaline protease diminishes the enterocyte damage associated with the systemic administration of recombinant mouse TNF-α. A mouse TNF-α-induced gastroenteritis model using markers of enterocyte apoptosis as a measure of the proinflammatory activity of the cytokine validates the utility of Aspergillus oryzae alkaline protease.

In addition, purified Aspergillus oryzae alkaline protease, when administered systemically to mice, protects against TNF-α-induced apoptosis of epithelial cells lining the terminal villi of the duodenum. The lesions induced in this animal model appeared first within cells that constitute the anatomical barrier between the host and the intestinal lumen. This is relevant for the development of IBD, as altered epithelial barrier function is a critical early step in disease pathogenesis.

In another embodiment, an affliction, ailment, condition, disease, disorder, illness, infirmity, inflammation, injury, malady, misery, pathosis, sickness, and/or syndrome mediated by TNF-α can be treated by administration of alkaline protease compositions as described herein. Further, an affliction, ailment, condition, disease, disorder, illness, infirmity, inflammation, injury, malady, misery, pathosis, sickness, and/or syndrome dependent upon TNF-α can be treated by administration of alkaline protease compositions as described herein. Further, an affliction, ailment, condition, disease, disorder, illness, infirmity, inflammation, injury, malady, misery, pathosis, sickness, and/or syndrome involving TNF-α can be treated by administration of alkaline protease compositions as described herein.

The preferred A. oryzae alkaline protease composition has the following properties: (1) resistance to inactivation by exposure to pH=2, pepsin, and trypsin; (2) heat stable at 37° C. for 3 hours; (3) ability to cleave and inactivate TNF-α; (4) stable in dry form at room temperature (˜25° C.) for 2 years; and (5) a pH optimum of from about pH˜6-10, most preferably from about pH˜7-10, most preferably at about pH˜8. The preferred alkaline protease acts therapeutically by means of decreasing or preventing TNF-α mediated inflammation.

### Alkaline Protease Activation of Beneficial Proteins

The alkaline protease described herein can be used individually or in combination to treat inflammatory processes that are known to involve distinct protein mediators. The action of alkaline proteases is not limited to inactivation, but can also be applied to the activation of beneficial proteins. Further, adverse effects can be limited through the use of a purified reagent with a limited range of activities in vivo. Additionally, purified proteases can be combined in various ratios to create known compositions with identifiable specificities for applications under selected conditions. The alkaline protease described herein specifically acts by means of cleaving and inactivating TNF-α, which may lead to the activation of beneficial proteins.

### Protease Effects on Gastrointestinal Function

Growth factors, cell surface receptors, extracellular matrix proteins, cell anchoring structures that maintain tissue organization, molecules that mediate cell migration within the tissues and cell-cell communication, nutrients, and host defense mediators are all candidate protein and peptide mediators of gastrointestinal physiology and disease, and may be acted upon by exogenous proteases provided in the diet or under a therapeutic regimen. The alkaline protease described herein specifically acts by means of cleaving and inactivating TNF-α, which may lead to the desired effects on gastrointestinal function.

Alternatively, proteases can activate or inactivate biologically important substrates in the gut lumen, at the surface of the mucosal epithelium or within GI-associated tissues. The alkaline protease can be used in purified form or in pre-determined compositions (mixtures), each of which have unique properties against specific substrates. Compositions of alkaline proteases have specific applications based on their unique synergistic compositions and stability properties in vivo.

### Proteases of Aspergillus oryzae

Four of the proteases produced by Aspergillus oryzae are listed in Table 1. They represent a range of catalytic types, including serine and metalloproteases. All belong to the endopeptidase category and in the aggregate act over a broad pH range. However, they differ in their pH optima, thermostability properties, and substrate specificities. The acid protease of A. oryzae (Table 1) exists in two similar, but disparate molecular forms, which have been shown to differ from one another in that the larger form is heavily glycosylated.

TABLE 1
Properties of Selected Proteases from Aspergillus oryzae

| | Deuterolysin | | | |
|---|---|---|---|---|
| | (Neutral | Alkaline | Serine | Aspergillopepsin O |
| Property | Protease II) | Protease | Carboxypeptidase | (Acid Protease) |
| Catalytic Type | Zn | Serine | Serine protease | Aspartic protease |
| | metalloprotease | protease | | |
| Reported | Hemoglobin | Hemoglobin, | | Hemoglobin |
| Substrate | (low), | Milk casein, | | Milk casein |
| Specificities^{a} | Milk casein | Salmon | | Bovine pancreatic |
| | (low), | protamine | | pepsinogen |
| | Salmon | sulfate, | | |
| | protamine | CBZ-val-leu- | | |
| | sulfate (high), | lys | | |
| | Boc-arg-val-arg- | | | |
| | arg | | | |
| pH optimum | 5.0–7.0 | 7.0–10.0 | 4.5 | 3.0–5.0 |
| Approx. MW | 19 kDa | 28 kDa | 67 kDa | 32 kDa (63 kDa |
| | | | | glycosylated form) |
| Amino acids | 177 | 282 | | |
| pI | 4.53 | 6.53 | | 3.9 and 3.5 |
| Heat stability | Stable at 100° C., | Stable at | | Unstable above 55° C. |
| | Unstable at | 37° C. for 3 h | | |
| | 75° C. | | | |
| EC Number | 3.4.34.39 | 3.4.21.63 | | 3.4.23.6 |

^{a}Relative activities against these natural and synthetic substrates vary widely.

### Alkaline Proteases Sources

The isolated alkaline protease can be purified from cells that naturally express it, purified from cells that have been altered to express it (recombinant), or synthesized using standard methods known in the art. The alkaline protease can also be obtained from commercial sources as a purified or isolated enzyme, in a cultural supernatant of Aspergillus oryzae, or as part of a food grade protease blend available from and Amano Enzyme Inc. (Elgin, Ill.). Purified strains of Aspergillus oryzae are also commercially available from American Type Culture Collection (ATCC) (Manassas, Va.). Alkaline protease from Bacillus spp. is available from Sigma Aldrich (St. Louis, Mo.) The strains may be grown by using standard methods in the art and the alkaline protease can be purified using methods described herein and as known in the art.

### Purification of Alkaline Proteases from Natural Sources

The isolated protease can be purified from cells that naturally express it using standard methods known in the art. A suitable protocol for purification is described in Examples 1 and 2 below, although the skilled person could readily develop alternative protocols based on standard biochemical principles. A protease according this invention should be “isolated” or “purified”. The proteases of the present invention can be purified to homogeneity or other intermediate degrees of purity. The level of purification will be based on the intended use. The critical feature is that the preparation allows for the desired function of the protease, even if in the presence of considerable amounts of other components. The preferred embodiment of an “isolated” or “purified” alkaline protease is where the alkaline protease component constitutes at least about 51% of the hydrolytic activity of the composition, measured against salmon protamine (nmoles Leucine/30 min./μg protein), and has a pH optimum equal to or greater than about pH˜8.0. Another embodiment of an “isolated” or “purified” alkaline protease is where it is the predominant protease in a composition, comprises at least 51% of the hydrolytic activity of the composition, wherein the specific activity of the “isolated” or “purified” alkaline protease has specific activity of at least about 900 for salmon protamine (nmoles Leucine/30 min./μg protein) and/or 100 for Val-leu-lys-pNA (mOD/min/μg protein). The specific activity may be determined by methods as taught herein and those methods known in the art.

### Preparation of Alkaline Proteases by Chemical Synthesis

The isolated protease can be synthesized using known protein synthesis methods such as solid-phase synthesis of polypeptides or chemical ligation. Other methods for chemical synthesis known in the art are described in Coligan et al. (Eds.) (2003) Current Protocols in Protein Science Wiley, which is incorporated by reference.

The language, “substantially free of chemical precursors or other chemicals”, includes preparations of the protease in which it is at least 95% free of chemical precursors or other chemicals that are involved in its synthesis.

### Preparation of Alkaline Proteases from Recombinant Sources

The isolated protease can be purified from cells that have been altered to express it (recombinant). DNA sequences encoding the protease may be inserted into an expression vector and then transformed (or transfected) in an appropriate host cell and/or expressed in a transgenic animal.

### Nucleic Acids

In particular, a nucleic acid molecule encoding the protease is cloned into an expression vector, the expression vector introduced into a host cell and the protein expressed in the host cell. The protease can then be isolated from the cells by an appropriate purification scheme using standard protein purification techniques which are well known to those of ordinary skill in the art and many are described in Sambrook et al. (2001) Molecular Cloning: A Laboratory Manual (3^{rd} Ed.) Cold Spring Harbor Laboratory Press, which is incorporated by reference. Other methods suitable for this invention which are known in the art are described in Ausubel et al. (2002) Short Protocols in Molecular Biology (5^{th} Ed.) Wiley, which is incorporated by reference, and New England BioLabs, Inc. (2004) 2005-2006 Catalog and Technical Reference, which is incorporated by reference.

The present invention further provides isolated nucleic acid molecules that encode a protease of the present invention (cDNA, transcript and genomic sequence). Such nucleic acid molecules will consist of, consist essentially of, or comprise a nucleotide sequence that encodes one of the alkaline protease of the present invention, an allelic variant thereof, or an ortholog or paralog thereof.

Moreover, an “isolated” nucleic acid molecule, such as a transcriptlcDNA molecule, is free of other nucleic acids from the biological species in which the nucleic acid occurs in nature. Preferably, an “isolated nucleic acid” is substantially free of other cellular material, or culture medium when produced by recombinant techniques, or chemical precursors or other chemicals when chemically synthesized. However, the nucleic acid molecule can be fused to other coding or regulatory sequences and still be considered isolated.

Accordingly, the present invention provides protease that comprises of the amino acid sequences provided in SEQ ID NO: 2. The alkaline protease may also comprise of the nucleic acid sequence which encodes SEQ ID NO: 2. A protein comprises of an amino acid sequence when the amino acid sequence is the final amino acid sequence of the protein. Accordingly, the present invention provides protease that consists of the amino acid sequences provided in SEQ ID NO: 2. The invention may also consist of the nucleic acid sequence which encodes SEQ ID NO: 2. A protein consists of an amino acid sequence when the amino acid sequence is the final amino acid sequence of the protein.

In another embodiment, sequence variants of at least 70% sequence homology with SEQ ID NO: 2 that retain the biological activity and substrate specificity of the alkaline protease are suitable for this invention. In another embodiment, sequence variants of at least 80%, at least 90%, at least 95%, at least 98%, and/or at least 99% sequence homology with SEQ ID NO: 2 that retain the biological activity and substrate specificity of the alkaline protease are suitable for this invention. The comparison of sequences and determination of percent identity and similarity between two sequences can be accomplished using a mathematical algorithm as described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1,Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991, all of the foregoing are incorporated by reference.

In another embodiment, sequence variants with over 70% sequence homology with SEQ ID NO: 2 that retain the biological activity and substrate specificity of the alkaline protease are suitable for this invention. In another embodiment, sequence variants with over 80%, with over 90%, with over 95%, with over 98%, and/or with over 99% sequence homology with SEQ ID NO: 2 that retain the biological activity and substrate specificity of the alkaline protease are suitable for this invention. The comparison of sequences and determination of percent identity and similarity between two sequences can be accomplished using a mathematical algorithm as described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991, all of the foregoing are incorporated by reference.

Further the genetic code is known to be redundant and several nucleic acids may code for SEQ ID NO: 2. Also, several nucleic acids may be deduced from the amino acid of SEQ ID NO: 2, preferably using the codon preference of the host organism. Any of these sequence variants of the alkaline protease as described herein and suitable for this invention retain the biological activity and substrate specificity of the alkaline protease as described herein.

### Vectors

The invention also provides vectors containing the nucleic acid molecules described herein. The term “vector” refers to a vehicle, preferably a nucleic acid molecule, which can transport the nucleic acid molecules. When the vector is a nucleic acid molecule, the nucleic acid molecules are covalently linked to the vector nucleic acid. The invention provides vectors for maintenance (cloning vectors) or vectors for expression (expression vectors) of the nucleic acid molecules. The vectors can function in prokaryotic or eukaryotic cells or in both (shuttle vectors). With this aspect of the invention, the vector includes a plasmid, single or double stranded phage, a single or double stranded RNA or DNA viral vector, or artificial chromosome, such as a BAC, PAC, YAC, or MAC. Plasmids suitable for this invention include but are not limited to pUC19, pBR322, pCMV, pSK Bluescript, pcDNA3, pcDNA3.1, pGEM, pGEX, pGST, pEGFP, and vectors disclosed in the Promega Complete Vector List, which is incorporated by reference.

The nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art. Appropriate cloning and expression vectors for prokaryotic and eukaryotic hosts, as well as protocols, are well known to those of ordinary skill in the art and many are described in Sambrook et al. (2001) Molecular Cloning: A Laboratory Manual (3^{rd} Ed.) Cold Spring Harbor Laboratory Press, which is incorporated by reference. Other methods suitable for this embodiment are known in the art described in Ausubel et al. (2002) Short Protocols in Molecular Biology (5^{th} Ed.) Wiley, which is incorporated by reference, and New England BioLabs, Inc. (2004) 2005-2006 Catalog and Technical Reference, which is incorporated by reference.

### Host Cells

The vector containing the appropriate nucleic acid molecule can be introduced into an appropriate host cell for propagation or expression using well-known techniques. Host cells may be prokaryotic, including but not limited to bacterial cells, or eukaryotic, including but not limited to insect, fungal, mold, yeast, animal, and/or plant cells. Several host cells suitable for this invention are described New England BioLabs, Inc. (2004) 2005-2006 Catalog and Technical Reference, which is incorporated by reference.

The recombinant host cells are prepared by introducing the vector constructs described herein into the cells by techniques readily available to the person of ordinary skill in the art. These include, but are not limited to calcium phosphate transfection, cationic lipid-mediated transfection, conjugation, DEAE-dextran-mediated transfection, electroporation, infection, lipofection, protoplast transformation, transduction, and other techniques such as those found in Sambrook et al. (2001) Molecular Cloning: A Laboratory Manual (3^{rd} Ed.) Cold Spring Harbor Laboratory Press, which is incorporated by reference, and Ausubel et al. (2002) Short Protocols in Molecular Biology (5^{th} Ed.) Wiley, which is incorporated by reference.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where the peptide is not secreted into the medium, which is typically the case with proteases, the protein can be isolated from the host cell by standard disruption procedures, including freeze thaw, sonication, mechanical disruption, and the use of lysing agents. The peptide can then be recovered and purified by purification methods known in the art including but not limited to acid extraction, affinity chromatography, ammonium sulfate precipitation, anion exchange chromatography, cationic exchange chromatography, high performance liquid chromatography (HLPC), hydrophobic-interaction chromatography, hydroxylapatite chromatography, lectin chromatography, and phosphocellulose chromatography.

### Alkaline Protease Polypeptides

The present invention provides for an alkaline protease. In particular, the invention also provides for an alkaline protease, preferably from Aspergillus oryzae. The alkaline protease described herein has the following properties, it is about 29 kDa in size as determined by SDS-PAGE gel (if glycosylated, the alkaline protease can be as large as about 36 kDa, and may be any size between 28-36 kDa), has a μl of about 6.5, has a pH optimum from about 7-10, and has substrate specificity of protamine >casein and a specific activity of at least 900 salmon protamine (nmoles Leucine/30 min./μg protein) and at least 100 Val-leu-lys-pNA (mOD/min/μg protein measured at 405 nm).

The alkaline protease as described herein predominately cleaves TNF-α, and poorly cleaves IFN-γ (as shown in FIGS. 11-12).

The present invention further provides proteases that consist essentially of the amino acid sequences provided in SEQ ID NO: 2 and are encoded by nucleic acids which encode for SEQ ID NO: 2. A protein consists essentially of an amino acid sequence when such an amino acid sequence is present with only a few additional amino acid residues, for example from about 1 to about 100 or so additional residues, typically from 1 to about 20 additional residues in the final protein. The present invention further provides proteins that comprise of the amino acid sequences provided in SEQ ID NO: 2, and are encoded by nucleic acids which encode for SEQ ID NO: 2. The present invention further provides proteins that consist of the amino acid sequences provided in SEQ ID NO: 2, and are encoded by nucleic acids which encode for SEQ ID NO: 2.

Allelic variants, paralogs, fragments, orthologs, and non-naturally occurring variants of the protease of the present invention can readily be generated using recombinant techniques. Such variants include, but are not limited to deletions, additions and substitutions in the amino acid sequence of the protease peptide. For example, one class of substitutions are conserved amino acid substitutions. Such substitutions are those that substitute a given amino acid in a protease peptide by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent is found in Bowie et al. (1990) Science 247:1306-1310, which is incorporated by reference. Any and all of the allelic variants, paralogs, fragments, orthologs, and non-naturally occurring variants of the protease as described herein retain the biological activity and substrate specificity of the alkaline protease.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. All of these methods are well known to those of ordinary skill in the art and many are described in Sambrook et al. (2001) Molecular Cloning: A Laboratory Manual (3^{rd} Ed.) Cold Spring Harbor Laboratory Press, which is incorporated by reference. Other methods known in the art are described in Ausubel et al. (2002) Short Protocols in Molecular Biology (5^{th} Ed.) Wiley, which is incorporated by reference, and New England BioLabs, Inc. (2004) 2005-2006 Catalog and Technical Reference, which is incorporated by reference.

Further in accordance with this invention, the protease may contain amino acids other than the 20 amino acids commonly referred to as the 20 naturally occurring amino acids and still retain the biological activity and substrate specificity of the protease. Further, many amino acids, including the terminal amino acids, may be modified by natural processes, such as processing and other post-translational modifications, or by chemical modification techniques well known in the art. Known modifications include, but are not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutarnate, formylation, gamma carboxylation, glycosylation, GPI anchor formation, hydroxylation, iodination, lipid attachment, methylation, myristoylation, oxidation, proteolytic processing, phosphorylation, prenylation, racemization, selenoylation, sulfation, transfer-RNA mediated addition of amino acids to proteins such as arginylation, and ubiquitination. The modifications as described herein and methods for adding them to the alkaline protease are known in the art as taught by Coligan et al. (Eds.) (2003) Current Protocols in Protein Science Wiley, which is incorporated by reference.

### Methods of Isolation of Alkaline Protease

The alkaline protease in this invention may be extracted, isolated, and/or purified from the host cells cultivated in a nutrient medium suitable for production of the protease using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the protease to be expressed and/or isolated. The cultivation takes place in a suitable nutrient medium comprising carbon and nitrogen sources and inorganic salts, using procedures known in the art. Suitable media are available from commercial suppliers or may be prepared according to compositions known in the art.

If the protease is secreted into the nutrient medium, the enzyme can be recovered directly from the medium. If the protease is not secreted, it is recovered from cell lysates. The cells expressing the alkaline protease is cultivated in a nutrient medium suitable for production of a polypeptide of interest using methods known in the art, many of which are described in Coligan et al. (Eds.) (2003) Current Protocols in Protein Science Wiley, which is incorporated herein by reference.

The protease may be recovered by methods known in the art including, but not limited to centrifugation, chromatography, evaporation, extraction, filtration, precipitation, preparative isoelectric focusing, and spray-drying. The recovered protease may then be further purified by a variety of chromatographic procedures including but not limited to affinity, chromatofocusing, gel filtration, hydrophobic, ion exchange, and size exclusion ion exchange. The recovered protease may then be further purified by a variety of electrophoretic procedures including but not limited to preparative isoelectric focusing (IEF). The recovered protease may then be further purified by a variety of differential solubility techniques including but not limited to ammonium sulfate precipitation. Other methods known in the art are described by Janson and Ryden (Eds) (1989) Protein Purification VCH Publishers, which is incorporated by reference. One suitable method for isolation of the alkaline protease is described in Examples 1-3.

The protease may be detected using methods known in the art that are specific for the protease. These detection methods may include use of specific antibodies, formation of an protease product, disappearance of an protease substrate, or SDS-PAGE. For example, an protease assay may be used to determine the activity of the protease. Procedures for determining protease activity are known in the art.

### Compositions

The proteases described herein may be included in compositions. These compositions may be pharmaceutical, nutritional supplements, food additive, foodstuff, aqueous solutions, and gels.

“Composition” as used herein, refers broadly to any composition containing a therapeutic agent and/or agents. The composition can comprise a dry formulation, an aqueous solution, a paste formulation, an organic solution formulation, a gel formulation, a jelly formulation, or a sterile composition. Compositions comprising the molecules described herein can be stored in freeze-dried form and can be associated with a stabilizing agent such as a carbohydrate.

The composition of the present preferred embodiment is orally administered in capsule (hard or soft, coated or uncoated), tablet (coated or uncoated), powder or granule (coated or uncoated) or liquid (solution or suspension) form and dissolves easily in the stomach.

The composition of the present invention may also include additional ingredients such as other enzymes, analgesics, vitamins, minerals, antioxidants, bioflavonoids, proanthocyanidins, herbs, herbal extracts, and plant and animal concentrates.

A present preferred embodiment may also contain an effective amount of a non-prescription or prescription analgesic, including but not limited to acetaminophen, acetylsalicylic acid (aspirin), ibuprofen, indomethacin, ketoprofen, naproxyn, salicylates, and sulindac. Dosages of such analgesics should not exceed federal regulations.

A present preferred embodiment may also contain an effective amount of vitamins, including but not limited to vitamin A, vitamin C, vitamin D, vitamin E, vitamin K, vitamin B1, vitamin B2, niacin, pantothenic acid, vitamin B6, folic acid, vitamin B12, and biotin.

A present preferred embodiment may also contain an effective amount of minerals, including but not limited to calcium, chloride, chromium, copper, fluoride, iodine, iron, magnesium, manganese, molybdenum, phosphorus, potassium, selenium, sodium, and zinc.

A present preferred embodiment may also contain an effective amount of antioxidants, bioflavonoids, and proanthocyanidins. The preferred antioxidant is vitamin C (ascorbic acid) is provided in the composition in the form of calcium ascorbate, which is a buffered form of ascorbic acid. Other potent antioxidants are in the form of bioflavonoids and proanthocyanidins, including bioflavonoids such as quercetin (3.3′,4′,5,7-pentahydroxyflavone) and rutin (3-rhamnoglucoside of 5,7,3′,4′-tetrahydroxyflavonol).

In aspects of the invention the starting material may contain added material which will improve the properties of the resulting dry protease containing powder or products resulting here from. Useful additives include materials selected from salts, inorganic minerals or clays, carbohydrates, coloring pigments, cellulose or derivatives thereof, biocides, dispersants, anti foaming agents, viscosity regulating agents, acid agents, alkaline agents, protease stabilizers, protease inhibitors, binders to other enzymes, and combinations thereof.

Calcium is provided in the present preferred embodiment as calcium citrate and calcium ascorbate because the mineral is optimally absorbed by the body from these two forms. Clinical experience with high doses of orally-administered microbial protease has resulted in muscle cramping in some patients. Oral administration of calcium in conjunction with microbial protease has alleviated the cramping.

### Pharmaceutical Compositions

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal,intrathecal, intravenous, oral, parenteral, rectally via an enema or suppository, subcutaneous, subdermal, sublingual, transdermal, and transmucosal. In addition, administration can by means of injection, tablet, pill, powder, liquid, gel, capsule, porous pouch, drops, patch, foams, or other means of administration.

A “pharmaceutical excipient” or a “pharmaceutically acceptable excipient” is a carrier, usually a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, 19^{th} Ed., Grennaro, A., Ed., 1995 which is incorporated by reference.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline protease can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

The preferred forms of administration in the present invention are oral forms know in the art of pharmaceutics. The pharmaceutical compositions of the present invention may be orally administered as a capsule (hard or soft), tablet (film coated, enteric coated or uncoated), powder or granules (coated or uncoated) or liquid (solution or suspension). The formulations may be conveniently prepared by any of the methods well-known in the art. The pharmaceutical compositions of the present invention may include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradermal), infusions, and combinations thereof.

Other compounds which can be included by admixture are, for example, medically inert ingredients, e.g., solid and liquid diluent, such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal clays; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, polysorbates or laurylsulphates; and other therapeutically acceptable accessory ingredients, such as humectants, preservatives, buffers and antioxidants, which are known additives for such formulations.

Liquid dispersions for oral administration can be syrups, emulsions or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

### Nutritional Compositions

The compositions of the alkaline protease described herein may be used in (or consumed in) nutritional supplements; dietary supplements; medical foods; nutriceuticals; food-stuffs such as pharmaceutical-benefit foods (e.g., “phoods”); beverages including traditional (e.g., regular oatmeal, whole-grain breads), fortified (e.g., orange juice with calcium); and “designer” (e.g., protein bars, smart spreads) products. The alkaline protease as described herein may be formulated in health bars, confections, animal feeds, cereals, yogurts, cereal coatings, foods, nutritive foods, functional foods, and combinations thereof.

### Dosages

The amount of active compound in the composition may vary according to factors such as the disease state, age, gender, patient history, risk factors, predisposition to disease, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

The dose range for adult human beings will depend on a number of factors including the age, gender, weight, and condition of the patient and the administration route. The dosages as suitable for this invention may be a composition, a pharmaceutical composition, or any other compositions described herein.

For each of the recited embodiments, the dose range for a patient can be 10, 20, 30, 40, 50, 60, 70, 80, 90, or 100 mg/kg and all increments therein. Preferably, the dose range for a patient can be 10-50 mg/kg and all increments therein. Alternatively, the dose range for a patient can be 25-50 mg/kg and all increments therein.

For each of the recited embodiments, the dose range for a patient can be 100, 200, 300, 400, 500, 600, 700, 800, 900, 1000, 1250, 1500, 1750, 2000, 2250, 2500, 2750, 3000, 3250, 3500, 3750, or 4000 mg per day and all increments therein. Preferably, the dose range for a patient is 1000 mg per day but can be 2000, 3000, or 4000 mg per day.

For each of the recited embodiments, the dose, if administered via injection (i.p.), can be 1, 2, 3, 4, 5, 6, 7, 8, 9, or 10 μg and all increments therein, per injection. Preferably, the dosage when administered-via injection is 10 μg.

For each of the recited embodiments, the dosage is typically administered once, twice, or thrice a day. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage may be administered for as long as symptoms persist. The patient can require “maintenance treatment” where the patient is receiving dosages every day for months, years or the remainder of their lives. In addition, the composition of this invention may be to effect prophylaxis of recurring symptoms. For example, the dosage can be administered once or twice a day to prevent the onset of symptoms in patients at risk, especially for asymptomatic patients.

For administration via injection, it is preferred that the treatment begin as a course of 4 injections at 0, 12, 24, and 36 hours. The injections then can continue once, twice, or thrice a day for as long as symptoms persist. Alternatively, the injections can be maintained to prevent the recurrence (or replace) of disease. Also, the injections can be administered as a prophylaxis for patients at risk, especially asymptomatic patients.

For-each of the recited embodiments, the dosage of alkaline protease may be adjusted based on specific activity, in particular, specific activity measured by cleavage of salmon protamine (nmoles Leucine/30 min./μg protein) and/or Val-leu-lys-pNA (mOD/min/μg protein). Preferably, pharmaceutical compositions of alkaline protease may comprise at least about 300, more preferably about 900 salmon protamine (nmoles Leucine/30 min./μg protein). However, pharmaceutical compositions of alkaline protease comprising at least about 30, or preferably about 100 Val-leu-lys-pNA (mOD/min/μg protein) measured at 405 nm, may be used.

The dosage can be administered as a single dose, a double dose, a triple dose, a quadruple dose, and/or a quintuple dose. The dosages can be administered singularly, simultaneously, and sequentially.

For each of the recited embodiments, the dosage of alkaline protease can be a therapeutically effective amount of alkaline protease, an amount effective for prophylaxis, and/or an amount effective for prevention. The dosage of alkaline protease can be an amount of alkaline protease effective to reduce inflammation, an amount effective to prevent inflammation, to reduce the severity -of inflammation, to eliminate inflammation, to slow the development of inflammation, to prevent the development of inflammation, and/or effect prophylaxis of inflammation.

The dosage form can be any form of release known to persons of ordinary skill in the art. The preferred dosage forms include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics, and combinations thereof is known in the art.

It will be appreciated that the pharmacological activity of the compositions can be monitored using standard pharmacological models that are known in the art. Furthermore, it will be appreciated that the inventive compositions can be incorporated or encapsulated in a suitable polymer matrix or membrane for site-specific delivery, or can be functionalized with specific targeting agents capable of effecting site specific delivery. For instance, the dosage form may be made such that it preferably releases in the duodenum, the jejunum, or the ileum. These techniques, as well as other drug delivery techniques are well known in the art.

### Routes of Administration

The compositions described herein may be administered in any of the following methods parenteral, intravenous, intraperitoneal, intramuscular, sublingual, buccal, or oral. The preferred routes of administration are buccal and oral. The administration can be local, where the composition is administered directly, close to, in the locality, near, at, about, or in the vicinity of, the site(s) of disease, e.g. inflammation or systemic, wherein the composition is given to the patient and passes through the body widely thereby reaching the site(s) of disease. Local administration can be administration to the cell, tissue, organ, and/or organ system which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral wherein the desired effect is systemic, composition is given by other routes than the digestive tract.

### EXAMPLES

The examples contained herein are offered by way of illustration and not by any way of limitation.

### Example 1

### Production of Alkaline Protease Powder

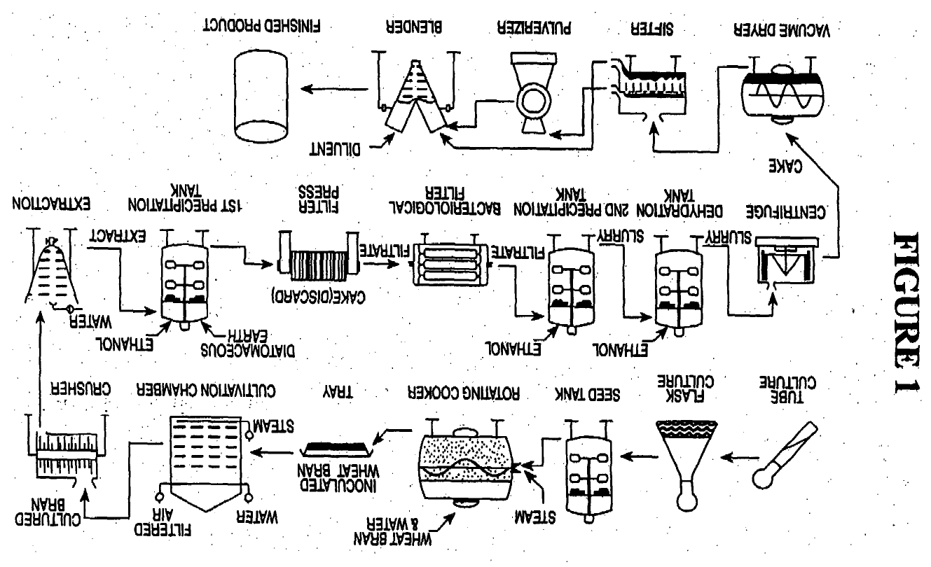

A method for producing alkaline protease from various species of Aspergillus fungi via solid state fermentation is illustrated diagrammatically in FIG. 1, although other production methods known in the art are also acceptable. Referring to FIG. 1, the fermentation process begins by taking a population of the desired fungi from a test tube culture and transferring it to a large flask for additional growth. This cultured fungi is then moved to a seed tank where it is further propagated. The resulting concentrated suspension of fungi is then transferred to a rotating cooker and mixed with sterilized koji (wheat, soy, or rice bran), water and steam where it is cooked for a sufficient period of time to inoculate the koji with fungi. The inoculated koji is then moved onto large trays, which are then transported to a cultivation chamber where the fungi are permitted to grow. Fermentation under controlled temperatures and humidity conditions may take from a few days to a week or more to complete.

At the conclusion of fermentation, the cultured koji is then transferred to a crusher device, which pulverizes the koji mash. The resulting pulverized mash is then moved to an extractor to filter the particulate matter from the slurry. For some processes, there may also be microfiltration or ultrafiltration steps to concentrate the aqueous enzymes before precipitation. The slurry is then moved to a first precipitation tank where it is mixed with ethanol and filtered through diatomaceous earth and then run through a filter press where the cake is discarded. The filtrate from the filter press is then processed through a bacteriological filter before it is moved to a second precipitation tank for further filtering and precipitation. The ethanol precipitation and bacteriological filter steps produce enzymes that are microbially very “clean” (e.g., they have very low microbes when compared to other food products such as fluid or pasteurized milk). The slurry is then centrifuged and the resulting cake is transferred to a vacuum dryer for drying. The dried proteinaceous material is then passed through a sifter and then a pulverizer to reduce the particle size. This material is then placed in a blender and diluent may or may not be added to standardize the potency of the finished powder product. This powder contains a heterogeneous mixture of microbial proteases, including some alkaline protease.

The protease powder prepared according to Example 1 is a preparation from A. oryzae is a heterogeneous preparation that cleaves salmon protamine sulfate, a common protease substrate, and inactivates mouse TNF-α by proteolysis. A commercially available version of this product, Protease 6.0, may be obtained from Amano Enzyme Co. Nagoya, Japan, for example.

TABLE 2
pH Activity Profile of Protease Powder prepared according
to Example 1

| pH | Activity (%) |
|---|---|
| 3 | 25 |
| 4 | 50 |
| 5 | 75 |
| 6 | 90 |
| 7 | 100 |
| 8 | 90 |
| 9 | 85 |
| 10 | 80 |
| 11 | 25 |

### Example 2

### Purification of Alkaline Protease from the Protease Powder Prepared According to Example 1

The product of Example 1 is similar to Protease 6.0 and it may be processed as described in this Example to purify alkaline protease.

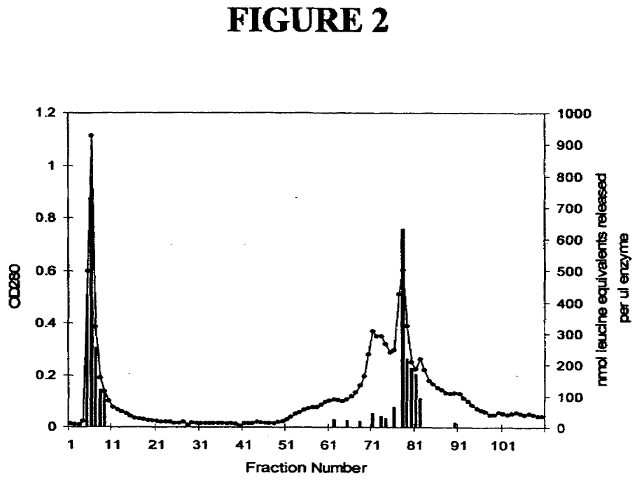

A solution of the protease powder prepared according to Example 1 was applied to the anion exchange resin Source 30Q™ (Pharmacia) at a semi-alkaline pH, and bound protein was eluted with a linear NaCl gradient. A significant portion of the protein did not bind to the ion exchange resin at pH˜8.0 (30Q Peak I) and contained substantial proteolytic activity (FIG. 2). These unbound fractions were pooled and retained for further fractionation.

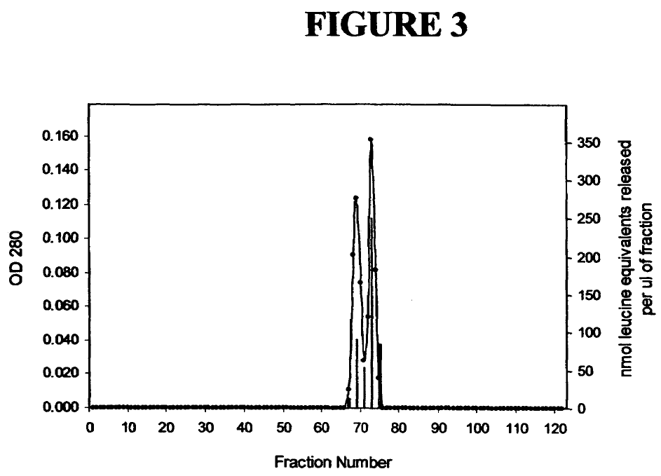

Among the bound material, a peak eluting at 250-280 mM NaCl at pH˜8.0 also contained activity (shaded area, FIG. 2). This material was designated as “30Q Peak IV,” and gel electrophoresis contained a single dominant protein species. Peak I from 30Q chromatography was then concentrated and applied to the cation exchange resin Source 30S at pH 5.5 in acetate buffer. Two protein peaks eluted at 10-60 mM NaCl in a linear NaCl gradient (FIG. 3), and both peaks had proteolytic activity against protamine (shaded bars). Peak II from 30S was further separated by gel filtration chromatography (FPLC on Superdex 75) and eluted as a single peak with an apparent molecular mass of 29 kDa.

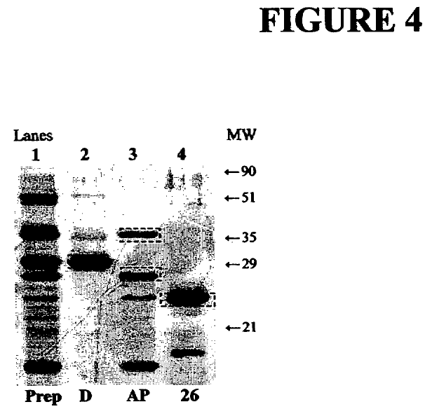

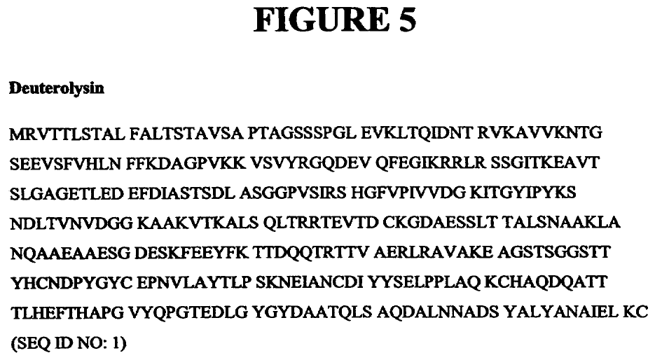

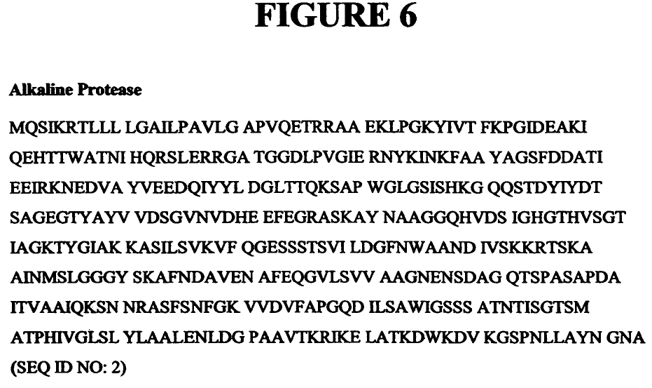

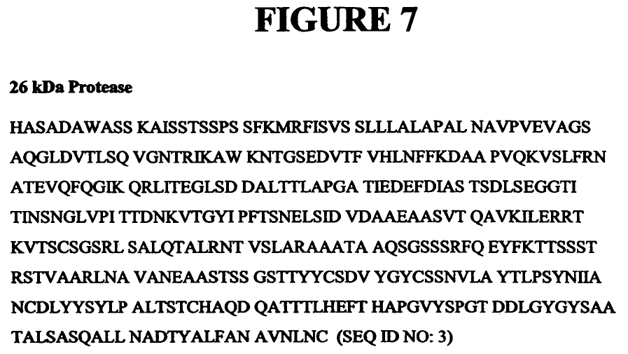

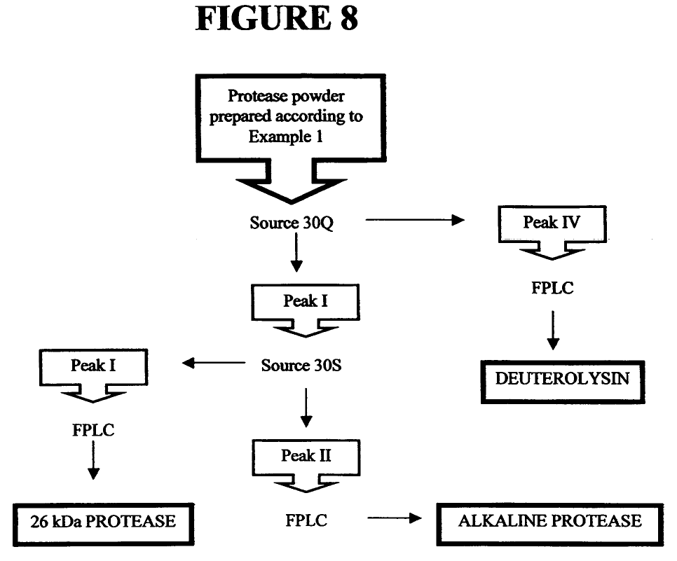

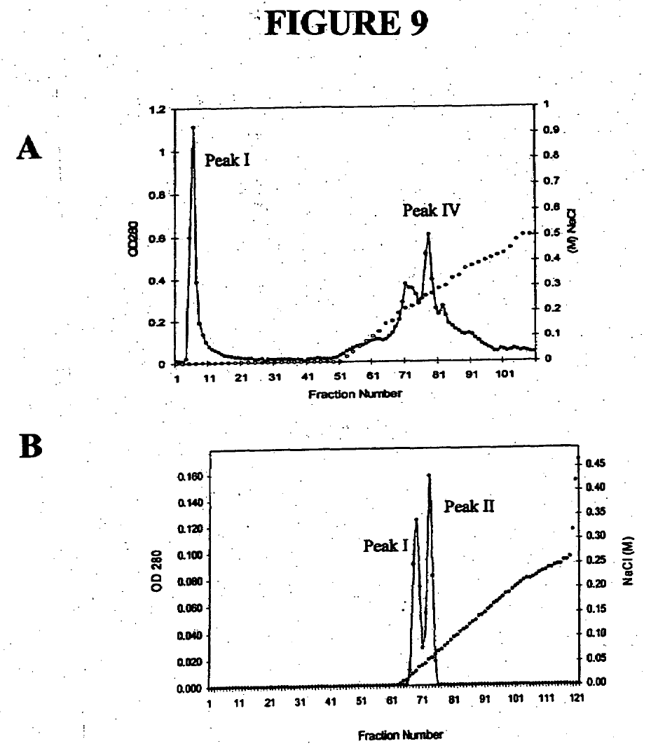

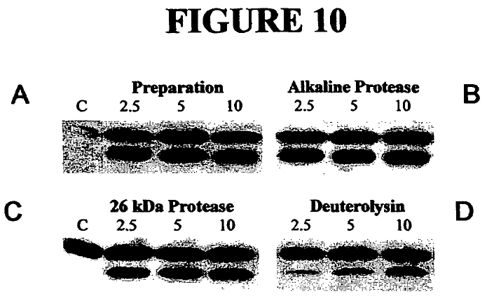

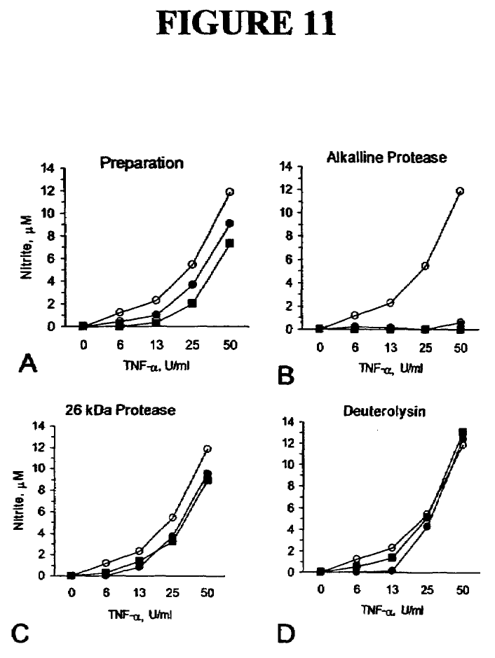

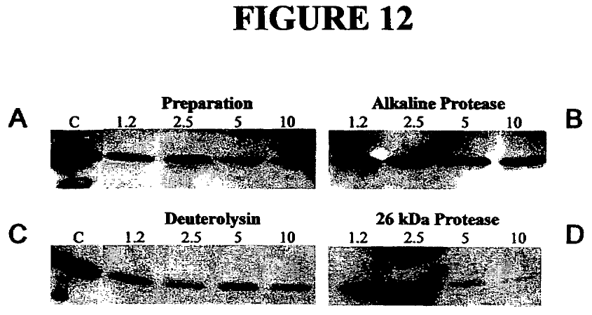

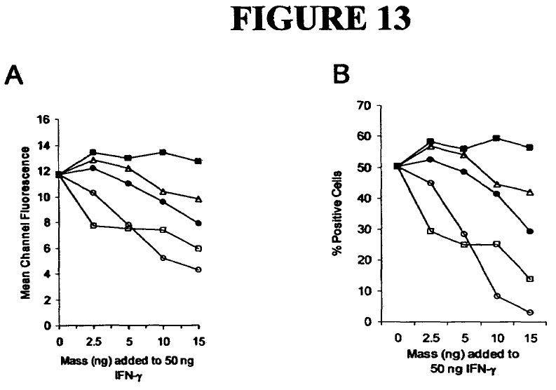

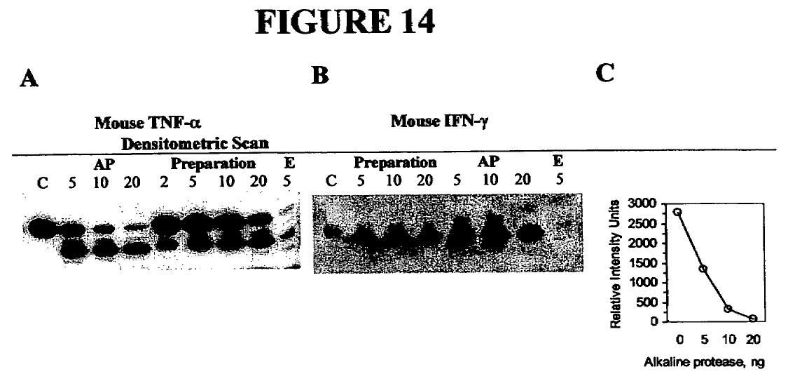

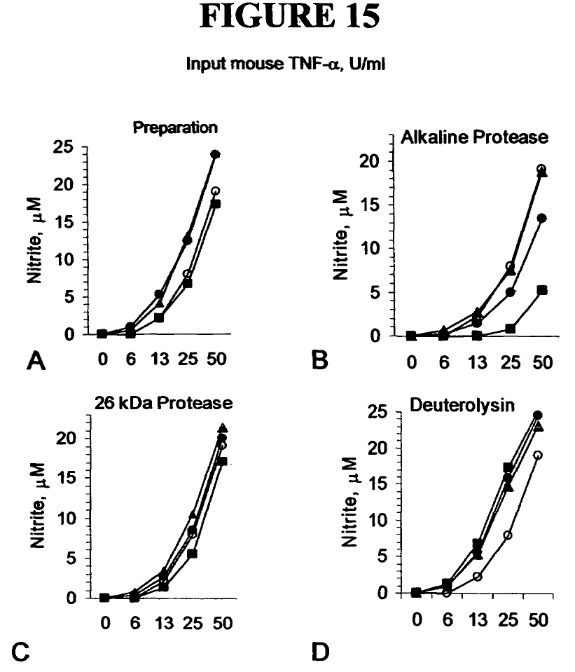

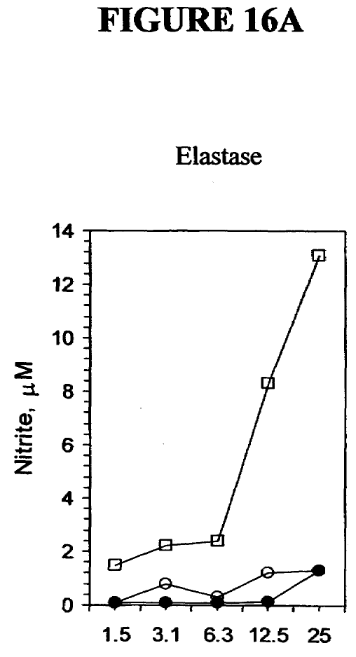

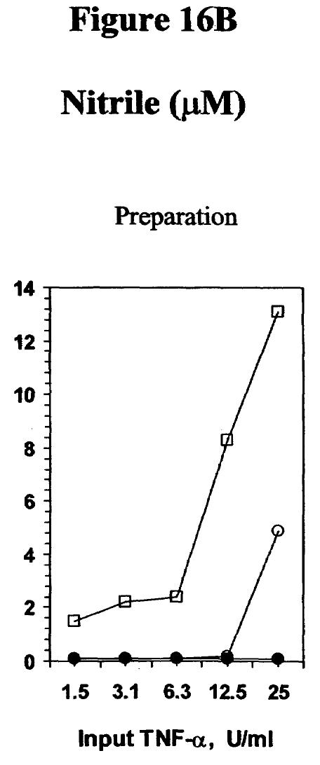

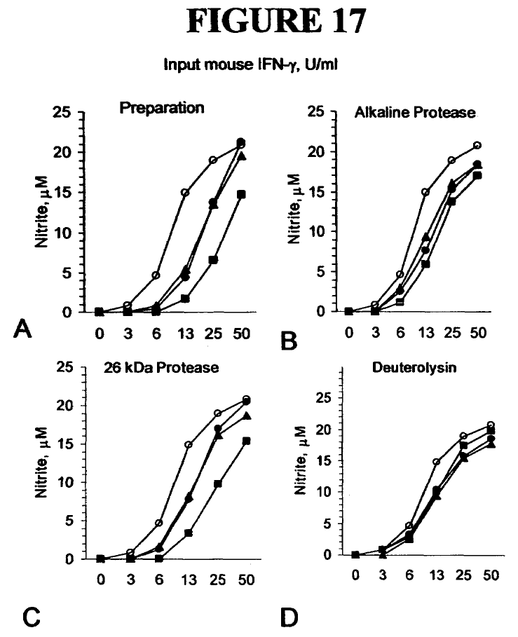

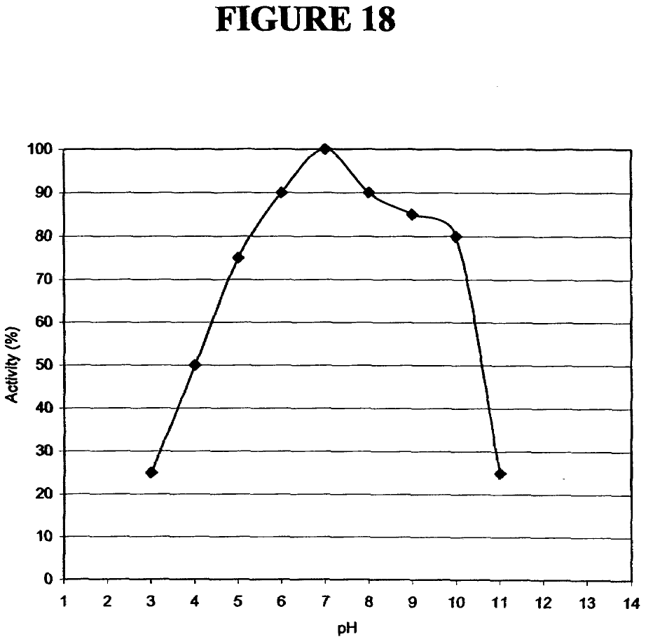

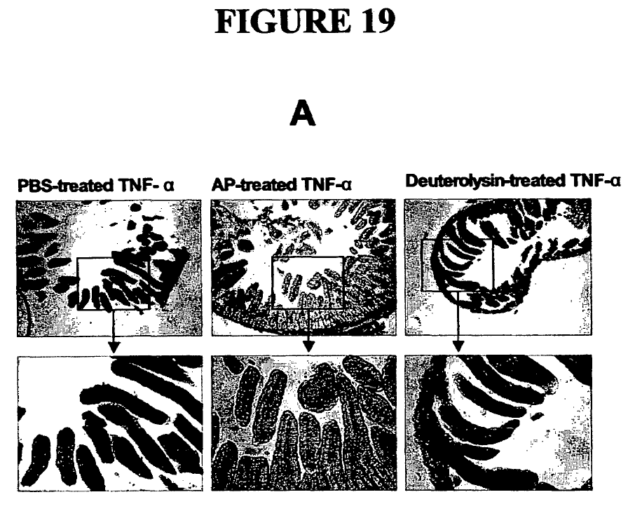

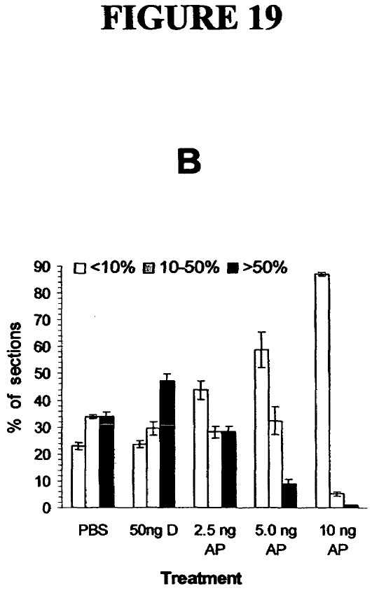

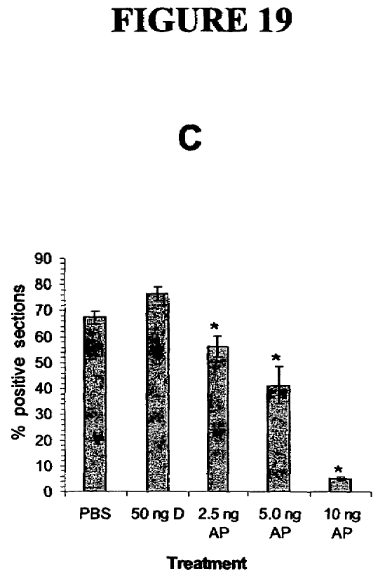

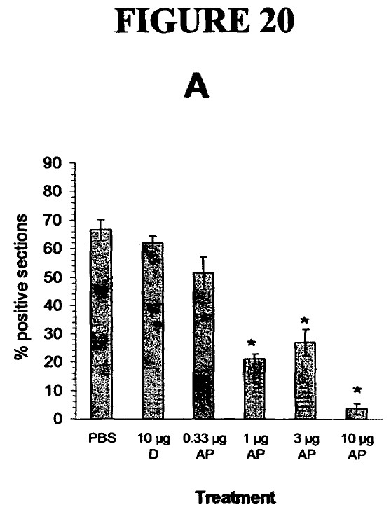

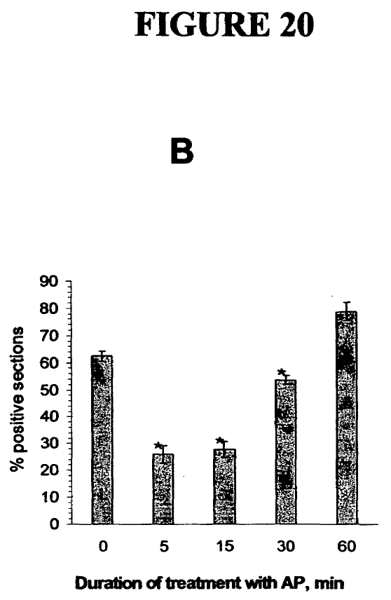

The starting material (the protease powder prepared according to Example 1) and the individual pooled fractions containing proteolytic activity were examined by sodium dodecyl sulfate-polyacrylamide gel electrophoresis (SDS-PAGE) under reducing conditions. Protein fractions were analyzed on a 12% SDS-PAGE gel and transferred either to Immobilon-P membranes for Western blot analysis with anti-AP or Immobilon-P^{SQ} (ISEQ 08100)(Millipore) for amino-terminal sequencing of selected bands. The sequencing was performed by the Molecular Biology Resource Facility at the University of Oklahoma Health Sciences Center (Oklahoma City, Okla.). The protease powder prepared according to Example 1 was found to contain no less than 20 distinct protein bands that could be stained with Coomasie Blue (Lane 1, FIG. 4). Peak IV from 30Q chromatography (lane 2) had a predominant species with an apparent molecular mass of 30 kDa. Lane 3 shows the appearance of 30S Peak II, which contained two major species (28 and 36 kDa) and a lower molecular mass peptide. Lane 4 was loaded with the first peak obtained from 30S chromatography and appeared as a single predominant species (26 kDa).

Thus, three distinct protein fractions with proteolytic activity were recovered from ion exchange chromatography and each contain unique predominant species when analyzed by SDS-PAGE. Each of the species identified in FIG. 4 by a dashed box was then recovered by SDS-PAGE followed by electrophoretic transfer to PVDF membranes. After staining the membranes, the selected protein bands were subjected to N-terminal amino acid sequencing. Note that two species from lane 3 were sequenced. The sequencing results are listed in Table 3.

TABLE 3
N-terminal amino acid sequencing of the proteases purified from the protease
powder prepared according to Example 1.

| | FIG. 4 lane | | N-terminal | Identical sequence |
|---|---|---|---|---|
| Fraction | number | Apparent Mr | Sequence | match with:^{a} |
| 30Q Peak IV | 2 | 30 kDa | TEVTDCKGD | A. oryzae Deuterolysin |
| | | | (SEQ ID NO: 4) | |
| 30S Peak II | 3 | 28 and 36 kDa | GLTTQKSAP | A. oryzae Alkaline Protease |
| | | | (SEQ ID NO: 5) | |
| 30S Peak I | 4 | 26 kDa | TKVTSNSGSR | A. oryzae Protease |
| | | | (SEQ ID NO: 6) | |

^{a}For the amino acid sequence of these proteins, see FIGS. 5–7.

The three proteases identified from the protease powder prepared according to Example 1 were deuterolysin (neutral protease II) (FIG. 5), alkaline protease (oryzin) (FIG. 6), and a protease cloned recently by Novozyme, labeled the “26 kDa protease.” (FIG. 7) The purified protease was identical with the Novozyme protein through 10 residues except at position 6, where an asparagine for cysteine substitution at position 6 was found. The purified 26 kDa protease sequenced also has significant sequence homology with two other proteins Novozyme has cloned from the Aspergillus species. Notice that the 28 and 36 kDa protein bands in lane 3 (alkaline protease) had the same N-terminal sequence. Alkaline protease has been cloned and has a predicted molecular mass of 29 kDa as determined by SDS-PAGE gel (Table 4).

TABLE 4
Biochemical properties of the proteases purified from the A. oryzae protease
powder prepared according to Example 1

| Property | Deuterolysin | Alkaline Protease | 26 kDa Protease |
|---|---|---|---|
| Calculated MW based | 19,017 | 29,084 | 18,528 |
| on sequence | | | |
| Apparent MW by | 29 kDa | 28 kDa; 36 kDa | 26 kDa |
| SDS-PAGE^{a} | | | |
| pI^{b} | 4.53 | 6.53 | 6.72 |
| Net charge at | −17 at pH 8.0 | +4.3 at pH 5.5 | +2.4 at pH 5.5 |
| fractionation pH^{b} | | | |
| Metal requirements | Zn, Ca, Co | — | Unknown |
| Heat stability | Stable at 90° C. for | Stable at 37° C. | Unknown |
| | 10 min | for 3 h | |
| pH optimum^{c} | pH 6–8 | pH 7–10 | 7.0 |
| Substrate specificity^{a} | Protamine > casein; | Protamine > casein, | protamine |
| | arg-val-arg-arg | hemoglobin; val-leu-lys | |
| | (SEQ ID NO: 7) | | |

^{a}Established by inventors.
^{b}This is the net charge of the protein at the pH of the buffer used to purify it to homogeneity (FIGS. 1 and 2).
^{c}pH optima vary with substrate.

Specific activity of the purified enzymes was determined by measuring hydrolysis of protamine sulfate and D-val-leu-lys-pNA. For the former, enzyme-substrate mixtures were prepared in 20 mM Tris-HCl, pH 7 or 8, and incubated for 30 min at 37° C. After precipitation with 14% trichloroacetic acid and removal of precipitated protein by centrifugation, the reaction mixtures were diluted in 500 mM sodium citrate buffer, pH 5, combined with 2% ninhydrin and boiled for 30 min. After dilution in 50% isopropanol, absorbance was measured at 570 nm wavelength, and results were referenced to leucine as a standard. Hydrolysis of D-val-leu-lys-pNA in 20 mM Tris-HCl buffer, pH 7, was determined by measuring absorbance at 405 nm wavelength every 10 sec over 10 min and calculating the change in absorbance (mOD units) per min per μg protein. Only the purified form of alkaline protease was found to cleave val-leu-lys peptide, and this protease was also slightly more active against protamine than the other enzymes (Table 5).

TABLE 5
Specific activities of the purified proteases of A. oryzae

| | | Alkaline | 26 kDa |
|---|---|---|---|
| Substrate | Deuterolys | Protease | Protease |
| Salmon protamine | 677 | 951 | 679 |
| (nmoles Leucine/ | | | |
| 30 min./μg protein) | | | |
| Val-leu-lys-pNA | 2.3 | 116.7 | 2.1 |
| (mOD/min/μg protein) | | | |

Overall, the results of these studies indicate that at least three major fungal proteases are present in substantial quantities in the protease powder prepared according to Example 1. These differ by a number of biochemical properties, including substrate specificity, and molecular mass.

TABLE 6
Scheme for Purification of Aspergillus oryzae Protease

| | Purification Details (See FIG. 8) |
|---|---|
| 1 | The protease powder prepared according to Example 1 is dissolved and dialyzed in 30 mM |
| | Tris, pH 8.0. An aliquot is retained for determination of total protein. |
| 2 | Conditions for Source 30Q chromatography. 3 mg the protease powder prepared |
| | according to Example 1 (based on protein, not weight) is loaded per ml of Source 30Q. |
| | Loading/running buffer: 30 mM Tris, pH 8.0 Salt gradient elution: 0–500 mM NaCI in |
| | 30 mM Tris, pH 8.0 |
| 3 | Elution profile for peak I and peak IV elution corresponds with FIG. 10A. |
| 4 | Concentrate and equilibrate Peak I from 30Q in 10 mM NaAc, pH 5.5. |
| 5 | Conditions for Source 30S chromatography |
| | 150 μg Peak I of 30Q is loaded per ml of Source 30S. |
| | Loading/running buffer: 10 mM NaAc, pH 5.5 |
| | Salt gradient elution: 0–250 mM NaCl in 10 mM NaAc, pH 5.5 |
| 6 | Elution profile for peak I and peak II elution corresponds with FIG. 10B. |
| 7 | The protease may be concentrated and equilibrated in 10 mM Tris, pH 7.0 and frozen. |
| 8 | FIG. 9 shows elution profiles from Source 30Q (A) and Source 30S (B). |

### Example 3

### Alkaline Protease Purity

The purity of an Aspergillus oryzae protease composition may be determined by SDS-PAGE and its activity against protamine sulfate, CBZ-val-leu-lys-NA, CBZ-arg-val-arg-arg-NA, AZCL-casein, mouse IFN-γ, and mouse TNF-α. Additional experiments on purity may be performed using antipeptide antibodies in Western blotting. The following parameters are met by highly purified proteases.
1. Apparent homogeneity (>98% in single peak) on FPLC or equivalent resolution gel permeation chromatography.
2. Homogeneous N-terminal amino acid sequencing provided through at least 8 residues.
3. Mass spectroscopy consistent with a single protein species, corresponding to the calculated molecular mass. Chromatography elution profiles (ion exchange and gel permeation) show a single peak.

All of these methods are well-known to those of ordinary skill in the art and many are described in Ausubel et al. (Eds.) (2002) Short Protocols in Molecular Biology (5^{th} Ed.) John Wiley & Sons, Inc., which is incorporated by reference, and Harris et al. (Eds.) (2006) Cell Biology Protocols John Wiley & Sons, Inc., which is incorporated by reference.

### Example 4

### Protease Stability

For determining the stability of the alkaline protease in drinking water over 0-4 days, proteases can be dissolved in autoclaved tap water at a concentration of 1 mg/ml and stored at either 25° C. or 4° C. At regular intervals (every 3 hours at 0-18 hours, every 8 hours at 18-24 hours, and every 12 hours at 24-96 hours), aliquots can be ascetically removed and tested for their ability to cleave protamine sulfate using a modification of the ninhydrin assay of Rosen. Results are expressed as % residual activity measured immediately after preparation of the protease solutions. In the instant invention, the catalytic activity of the purified alkaline protease is stable with 4° C. storage for several weeks. The alkaline protease as described herein is soluble in water and stable (retention of >80% of its activity for 2 days).

### Example 5

### Alkaline Protease Activity Assay

Reagents

Protamine sulfate from salmon (Sigma, St. Louis, Mo.): 1% (10 mg/ml) solution in reaction buffer. Fushima et al use 2% and denature the protein by boiling for 30 min in 100 mM phosphate buffer, pH 7.0. (Sigma indicates that this product has a solubility limit of 10 mg/ml.)

Leucine (Sigma, St. Louis, Mo.): Prepare 10 mM stock (1.31 mg/ml) in reaction buffer and dilute to titratable range (10-100 μM) for standard curve (See Table 1; Rosen, 1967).

Buffers and Solutions

Phosphate Buffer, 100 mM pH 7.0

Sodium Citrate Buffer, 0.5M pH 5.0

Trichloroacetic Acid, 14% solution in water (140 g/l)

1:1 mixture of 2-propanol (isopropanol) and water.

Procedure
1. In 1.5 ml Eppendorf tubes, combine test enzyme+reaction buffer up to 200 μl. Add 200 μl of substrate (1% protamine).
2. Incubate the mixture at 37° C. (or a temperature optimal for a given enzyme). Alternatively, one can incubate for various times if a kinetic measurement is to be made.
3. Terminate the reaction by the addition of 400 μl 14% TCA, vortex mix and incubate on ice for 30 min.
4. Remove precipitated proteins by centrifugation at 10,000 rpm for 15 min at 4° C.
5. Recover 100 μl of each sample and combine with 200 μl Sodium Citrate Buffer, pH 5.0 in a 12×75 polypropylene tube. Mix well.
6. Add 100 μl of a 3% solution of Ninhydrin (Sigma Chemical, St. Louis, Mo.). Mix, cover the tubes with marbles and place in a boiling water bath for 10 min.
7. Cool the mixtures on ice and then add 1 ml of 2-propanol/water mixtures. Shake vigorously and allow cooling at room temperature.
8. Read Absorbance at 570 nm (purple color) and calculate nanomoles by reference to a standard curve prepared with leucine. Dilute samples if necessary to achieve A₅₇₀<0.8. (Color should be stable for several hours.)
9. Activity of enzyme is expressed as nanomoles of leucine equivalents released per milligram of protein (endpoint assay) or nanomoles of leucine equivalents/min/μg protein (kinetic assay).
10. Controls include the following.
   a. Buffer control (no enzyme, no substrate)
   b. No incubation control (all components mixed and then immediately precipitated with TCA)
   c. Substrate control (w/o enzyme; incubated and the TCA precipitated)
   d. Enzyme Control (w/o substrate; incubated and the TCA precipitated)

### Example 6

### Acid Protease Activity Assay

The fungal protease powder prepared according to Example 1 contains both endo-peptidase and expo-peptidase activities, as well as significant amounts of starch-saccharifying activities.

Assay Principle

This assay is based on 30 minute enzymatic hydrolysis of a hemoglobin substrate at pH 4.7 and 40° C. Unhydrolyzed substrate is precipitated with trichloracetic acid and removed by filtration. This assay is useful for determining the proteolytic activity, expressed as hemoglobin units on the on the tyrosine basis (HUT) at pH 4.7, of protease powder preparations and subsequently purified fractions thereof. In particular, this assay is useful for determining the proteolytic activity at pH 4.7, expressed in HUT, of protease powders and subsequently purified fractions derived from Aspergillus oryzae and Aspergillus niger. This assay is useful for determining the proteolytic activity at pH 4.7, expressed in HUT, for any given protease.

Assay Apparatus
1. Constant Temperature Bath (40° C.+0.1° C.)
2. Spectrophotometer capable of measuring samples at least about 275 nm
3. Standardized pH Meter

Assay Reagents and Solution

Acetate Buffer Solution

Dissolve 136 grams sodium acetate trihydrate in sufficient water to make 500 mL. Mix 25 mL of this solution with 50.0 mL of 1M acetic acid, dilute to 1000 mL with water and mix. The pH of this solution should be at least about 4.7 (±0.02).

Substrate Solution

Weigh 4.0 grams of hemoglobin into a 250 mL beaker. With continuous stirring, add 100 ml of distilled water to which 1-2 drops of antifoam have been added. Stir for 10 minutes and adjust the pH 1.0±1.7 with 0.3 N hydrochloric acid. After 10 minutes, adjust the pH to 4.7 by adding 0.5 N sodium acetate. Quantitatively transfer the solution to a 200 mL volumetric flask and dilute to volume with distilled water. This solution is stable for about 5 days at around 4° C.

Trichloracetic Acid (TCA) Solution

Dissolve 140 g of TCA in about 750 ml of water. Transfer the solution to a 1000 mL volumetric flask, dilute to volume with water, and mix thoroughly.

Sample Preparation

Dissolve an amount of the sample in the Acetate Butter Solution to produce a solution comprising between 9 and 22 HUT/mL. (This concentration will produce an absorbance reading, in the procedure below, within the preferred range of 0.2 and 0.5).

Assay Procedure
1. Pipet 10 mL of the substrate solution into a series of test tubes. It is recommended to prepare three test tubes for each sample (2 for the reaction mixture, and 1 for the enzyme blank) and 1 test tube for substrate blank. Stopper the tubes and heat in a water bath at 40° C. for about 5 minutes.
2. Prepare the reaction mixtures by adding 2.0 mL of the sample preparation to the equilibrated substrate. Begin timing the reaction the moment the solution added. Mix by vortexing, stopper, and return to the 40° C. water bath.
3. Prepare the substrate blank by adding 2.0 mL of the Acetate Buffer Solution to the substrate blank tube. Begin timing the reaction the moment the solution is added. Mix by vortexing, stopper, and return to the 40° C. water bath.
4. After 30 minutes, add 10.0 mL of the Trichloracetic Acid Solution to each tube and shake vigorously against the stopper for about 40 seconds. Allow the test tubes to cool at room temperature (by convention 25° C.) for 1 hour, shaking each tube against the stopper every 10-12 minutes during this period.
5. Prepare the enzyme blanks by adding 10.0 mL of the Trichloracetic Acid Solution to 10.0 mL of the equilibrated substrate and shake well for 40 seconds. To this mixture add 2.0 ml of the Sample Preparation that has been heated in the 40° C. water bath for 5 minutes. Cool at room temperature for 1 hour, shaking the test tubes at 10 to 12 minute intervals.
6. At the end of 1 hour, shake each tube vigorously, and filter through 11 cm Whatman No. 42 filter paper (or equivalent) refiltering the first half through the same filter paper.
7. Determine the absorbance of each filtrate in a 1 cm cell at 275 nm, with a suitable spectrophotometer, using the filtrate from the substrate blank to zero the instrument.
8. Correct each reading by subtracting the appropriate enzyme blank reading and record the value so obtained as AU (Arbitrary Units).

If a corrected absorbance reading between 0.2 and 0.5 is not obtained, repeat the test using more less of the Enzyme Preparation as necessary.

Standard Curve

Transfer 100.0 mg of L-Tyrosine, chromatographic-grade (or equivalent) (Sigma-Aldrich Chemical Co., St. Louis), previously dried to constant weight, to a 1000 ml volumetric flask. Dissolve in 60 ml of 0.1 N hydrochloric acid. When the L-Tyrosine is completely dissolved, dilute the solution to volume with water, and mix thoroughly. This solution contains 100 μg of tyrosine per 1.0 ml. Prepare three more dilutions from this stock solution to contain 75.0, 50.0 and 25.0 μg of tyrosine per mL. Determine the absorbances of the four solutions at 275 nm in a 1-cm cuvette on a suitable spectrophotometer in comparison to 0.006 N hydrochloric acid.

Prepare a plot of absorbance per μg of tyrosine. Multiply this value by 1.10, and record it as “As”. A value of approximately 0.0084 should be obtained.

Calculations

HUT/g=((Au/As)×22/30w) where:
Au=Absorbance of enzyme filtrate at 275 nm
As=Slope of the curve of Abs.-vs.-μg Tyrosine×1.10
W=Weight, in grams, of enzyme added in the 2.0 ml aliquot
22=Final volume of the test solution: 30=Reaction time (minutes)

### Example 7

### Cleavage and Inactivation of Human Cytokines by Three Aspergillus oryzae Proteases

### Human TNFα Hydrolysis

Human TNF-α is cleaved and inactivated by Aspergillus oryzae alkaline protease. Purified cytokine (50 ng) was first combined with the following amounts [2.5, 5, and 10 ng] of protease powder prepared according to Example 1 (preparation), as well as alkaline protease, 26 kDa protease, and deuterolysin prepared according to Example 2. Each mixture was incubated overnight at 37° C. and examined by Western blotting for evidence of proteolysis. (FIG. 10A-D) The protease powder produced according to Example 1 cleaved human TNF-α to produce a fragment approximately 1-2 kDa smaller than the native cytokine. Alkaline protease and, to a lesser extent, 26 kDa protease each produced a similar pattern of cleavage, while deuterolysin was without significant effect. Because the antibody used in this Western blot was specific for the carboxyl terminus of human TNF-α, these enzymes cleaved the cytokine at the amino terminus yielding a smaller peptide that retained the C-terminal epitope.

### Human TNF-α Activity

Protease-treated human TNF-α was then tested for its bioactivity in the C2C12 cell assay. The results are shown in FIG. 12 and indicate that A. oryzae alkaline protease selectively inactivated human TNF-α, while the other proteases had only a limited effect. Although the 26 kDa protease partially digested human TNF-α, it did not significantly alter the biological activity of the cytokine. (FIG. 12A-D)

### Human IFN-γ Degradation

While alkaline protease appeared to be quite active against human TNF-α, the protease did not cleave human IFN-γ. (FIG. 12A-D) By contrast, both deuterolysin and 26 kDa protease cleave human IFN-γ. Cleavage in this experiment was evidenced by the disappearance of the native 17 kDa IFN-γ band, rather than the appearance of distinct cleavage peptides. Because the antibody used in this Western blot was specific for the carboxyl terminus of human IFN-γ, these enzymes attack the cytokine at the C terminus (e.g., there is a loss in reactivity with the anti-cytokine antibody, rather than the appearance of smaller peptide products)

FIG. 13A-B shows the effects of the purified A. oryzae proteases on the Fc receptor-inducing activity of human IFN-γ. Samples of the cytokine were first treated with the indicated Pseudomonas or fungal proteases: Elastase (∘); Preparation (); Alkaline Protease (□); Deuterolysin (▪); and 26 kDa Protease (Δ). The treated samples were added to human promyelocytic U937 cells to induce expression of Fc receptors for IgG, which was detected by flow cytometry. The results are plotted either as a decrease in of mean channel fluorescence (A) or a decrease in % positive cells (B) plotted against increasing protease amounts.

### Example 8

### Cleavage and Inactivation of Mouse Cytokines by Purified Aspergillus oryzae Alkaline Protease, Deuterolysin, and 26 kDa Protease

Hydrolysis of Murine TNF-α

The protease powder prepared according to Example 1 inactivates mouse recombinant TNF-α by limited proteolysis. (FIG. 16A-B) This property was established by measuring the loss of activity of TNF-α in a bioassay in which the cytokine was used to coactivate mouse myoblast C2C12 cells for the production of NO by transcriptional activation of the inducible NO synthase (iNOS) gene. The assay can be used to measure either TNF-α or IFN-γ activity by adding test samples containing one of the cytokines to cells stimulated with an excess of the other cytokine. Nitrite is a stable endproduct of NO produced by the cells. Using Pseudomonas elastase as a positive control, these data show that the protease powder prepared according to Example 1 substantially reduced the bioactivity of TNF-α in this assay. (FIG. 16A-B).

The protease powder prepared according to Example 1 also caused a demonstrable cleavage of mouse TNF-α as revealed by Western blotting. (FIG. 14A-C) In this experiment, the protease powder prepared according to Example 1 also cleaved both mouse TNF-α and mouse IFN-γ yielding products with molecular masses only slightly less than those of the native cytokines. These principal peptide products were surprisingly resistant to further proteolytic cleavage. Of interest, alkaline protease (AP) purified from the protease powder prepared according to Example 1 evidenced the same substrate specificity. Because the anti-TNF-α used here was specific for an epitope located in the carboxyl terminus of the cytokine, the pattern of cleavage is most consistent with attack at the amino terminus of TNF-α (e.g., there is a loss of molecular mass without a loss of the epitope recognized by the antibody).

### Murine TNF-α Activity

The three purified proteases were then compared for their ability to inactivate mouse TNF-α using the C2C12 cell bioassay. Two principal findings are apparent from this analysis, which are shown in FIG. 16A-B. First, alkaline protease showed a dose-dependent inactivation of mouse TNF-α as reflected by the reduced ability of protease-treated TNF-α to co-activate C2C12 cells for NO (nitrite) production. While the 26 kDa protease appeared to be devoid of such activity, deuterolysin actually enhanced NO production slightly.

### Murine IFN-γ

To evaluate the specificity of this effect, the three Aspergillus oryzae proteases were tested for their ability to inactivate mouse IFN-γ, and a different pattern was seen. As shown in FIG. 17, the protease powder prepared in according to Example 1 showed some activity against the cytokine, and this was primarily associated with the 26 kDa protease. Alkaline protease did not significantly affect the activity of mouse IFN-γ. This was an unexpected finding given the ability of alkaline protease to cause partial proteolysis of IFN-γ. (FIG. 17) Thus, alkaline protease partial degrades mouse IFN-γ without altering its bioactivity. These data demonstrate that the effects of alkaline protease on mouse TNF-α are specific for this cytokine and not seen when mouse IFN-γ was exposed to the enzyme.

Table 7 summarizes the results of these studies on human and mouse cytokines and suggests the following conclusions: (i) alkaline protease is the active component of the protease powder prepared according to Example 1 preparation that targets both mouse and human TNF-α; (ii) Alkaline protease is active against human IFN-γ and partially hydrolyzes mouse IFN-γ (FIG. 14A-C), but does not inactivate the mouse cytokine; (iii) mouse IFN-γ is somewhat susceptible to inactivation by 26 kDa protease (FIG. 17), although the protease does not inactivate human IFN-γ (FIG. 12).

These results indicate that the purified proteases of A. oryzae show both species and substrate specificity and, importantly, can inactivate an important proinflammatory cytokine, TNF-α. Therefore alkaline protease is potent anti-inflammatory agent with therapeutic utilities.

TABLE 7
Summary of the effects of A. oryzae Proteases on Mouse
and Human Cytokines

| | Human | Human | Mouse | Mouse |
|---|---|---|---|---|
| Enzyme | TNF-α | IFN-γ | TNF-α | IFN-γ |
| The protease | + | + | + | +/− |
| powder prepared | | | | |
| according to | | | | |
| Example 1 | | | | |
| Alkaline Protease | + | + | + | −^{a} |
| Deuterolysin | − | − | − | − |
| 26 kDa Protease | −^{a} | − | − | +/− |

+ Indicates that the protease inactivated the cytokine.
^{a}Cleavage of the cytokine occurred without inactivation.

### Example 9

### Effects of AP on TNF-α-Initiated Inflammation in vivo.

TNF-α is a potent mediator of cell death in vivo and has been shown to cause apoptosis in intestinal mucosal epithelial cells when administered systemically. A series of experiments was performed in which the activation of caspase-3 within the intestine was measured. Preliminary experiments showed that 5-10 ng of TNF-α injected i.p. was sufficient to induce the expression of activated caspase-3 in the villous epithelium of the duodenum. To determine whether AP blocked TNF-α activity in this in vivo assay, the cytokine was first treated either with PBS, AP or D for 2 h, the reaction mixtures were inactivated by the addition of FBS, and the cytokine-protease mixtures were injected i.p. into mice. One hour later the mice were euthanized, samples of their intestines were collected and fixed, and tissue sections were analyzed by immunohistology for the expression of activated caspase-3 (FIG. 19A). The results are shown in FIG. 22 in which data are expressed in two forms, either as the frequency distributions of sections expressing different levels of activated caspase-3 (FIG. 19B) or as the percentages of tissue sections in which 10% or more of the villi stained for activated caspase-3 (FIG. 19C). Treating TNF-α with increasing concentrations of AP, but not D, destroyed the ability of the cytokine to activate caspase-3 in intestinal epithelial cells.

A modification of this experiment was then performed by injecting various amounts of the proteases for 15 min prior to challenge with TNF-α (FIG. 20). Intestinal tissues were again recovered 1 h later and the expression of activated caspase-3 was measured. Pretreating mice with AP at doses of 1-10 μg blocked the action of TNF-α in vivo, whereas pretreatment with D was without a significant inhibitory effect (FIG. 20A). However, the ability of AP to provide protection against the apoptotic effects of TNF-α in vivo was limited in its duration (FIG. 20B). Thus, pretreatment of the mice for up to 30 min prior to TNF-α challenge prevented caspase-3 activation, whereas delaying cytokine challenge beyond 30 min diminished the protective effect of the protease.

Mice were injected i.p. with 5 ng of recombinant mouse TNF-α or protease-treated TNF-α, and 2 cm segments of their duodenums were collected 1 h later. The tissues were fixed with 4% paraformaldehyde and 5 μm thick sections were prepared and stained for activated caspase-3 by immmunoperoxidase techniques as known in the art. The sections were first treated with rabbit antibody to activated (cleaved) mouse caspase-3, and bound antibody was detected using a Histostain-SP kit. For each mouse, tissues were scored by enumerating the number of sections in which less than 10%, 10-50%, or >50% of the intestinal villi expressed activated caspase-3. At least 48 sections were analyzed in this fashion from each mouse.

### Example 10

### Treatment of Ulcerative Colitis

A human patient diagnosed with ulcerative colitis can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 11

### Treatment of Ulcerative Colitis

A human patient diagnosed with ulcerative colitis can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment according to the lessening of the symptoms and/or the development of side effects.

### Example 12

### Treatment of Ulcerative Colitis

A human patient diagnosed with ulcerative colitis can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 13

### Treatment of Ulcerative Colitis

A 70 kg human patient diagnosed with ulcerative colitis can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 14

### Treatment of Ulcerative Colitis

A human patient diagnosed with ulcerative colitis can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 15

### Treatment of Ulcerative Colitis

A 70 kg human patient diagnosed with ulcerative colitis can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 16

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 25-50 mg/kg of alkaline protease every day for 7-10 days. The human patient is monitored and treatment continued accordingly to prevent the occurrence of ulcerative colitis.

### Example 17

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment continued accordingly to prevent the occurrence of ulcerative colitis.

### Example 18

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 25-50 mg/kg of alkaline protease every week for 6 weeks. The human patient is monitored and treatment continued accordingly to prevent the occurrence of ulcerative colitis.

### Example 19

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising 1750-3500 mg of alkaline protease every day for 7-10 days. The human patient is monitored and treatment continued accordingly to prevent the occurrence of ulcerative colitis.

### Example 20

### Prophylaxis of Ulcerative Colitis

A human patient diagnosed with risk factors for ulcerative colitis can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treatment continued accordingly to prevent the occurrence of ulcerative colitis.

### Example 21

### Prophylaxis of Ulcerative Colitis

A human patient diagnosed with risk factors for ulcerative colitis can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treatment continued accordingly to prevent the occurrence of ulcerative colitis.

### Example 22

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 23

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 24

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 25

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 26

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 27

### Prophylaxis of Recurrence or a Relapse of Ulcerative Colitis

A human patient previously diagnosed with ulcerative colitis who is not instantly experiencing any symptoms can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 28

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 29

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 30

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 31

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 32

### Treatment of Inflammatory Bowel Disease (IBD)

A human patient diagnosed with inflammatory bowel disease (IBD) can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 33

### Treatment of Inflammatory Bowel Disease (IBD)

A human patient diagnosed with inflammatory bowel disease (IBD) can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 34

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days. The human patient is monitored and treatment continued accordingly to prevent the occurrence of ulcerative colitis.

### Example 35

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment continued accordingly to prevent the occurrence of ulcerative colitis.

### Example 36

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 25-50 mg/kg of alkaline protease every week for 6 weeks. The human patient is monitored and treatment continued accordingly to prevent the occurrence of ulcerative colitis.

### Example 37

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising 1750-3500 mg of alkaline protease every day for 7-10 days. The human patient is monitored and treatment continued accordingly to prevent the occurrence of ulcerative colitis.

### Example 38

### Prophylaxis of Inflammatory Bowel Disease (IBD)

A human patient diagnosed with risk factors for inflammatory bowel disease (IBD) can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treatment continued accordingly to prevent the occurrence of ulcerative colitis.

### Example 39

### Prophylaxis of Inflammatory Bowel Disease (IBD)

A human patient diagnosed with risk factors for inflammatory bowel disease (IBD) can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treatment continued accordingly to prevent the occurrence of ulcerative colitis.

### Example 40

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 41

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 42

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 43

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 44

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 45

### Prophylaxis of Recurrence or a Relapse of Inflammatory Bowel Disease (IBD)

A human patient previously diagnosed with inflammatory bowel disease (IBD) who is not instantly experiencing any symptoms can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 46

### Treatment of Crohn's Disease

A human patient diagnosed with Crohn's disease can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 47

### Treatment of Crohn's Disease

A human patient diagnosed with Crohn's disease can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 48

### Treatment of Crohn's Disease

A human patient diagnosed with Crohn's disease can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 49

### Treatment of Crohn's Disease

A human patient diagnosed with Crohn's disease can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 50

### Treatment of Crohn's Disease

A human patient diagnosed with Crohn's disease can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 51

### Treatment of Crohn's Disease

A human patient diagnosed with Crohn's disease can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

### Example 52

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 25-50 mg/kg of alkaline protease every day for 7-10 days. The human patient is monitored and treatment continued accordingly to prevent the occurrence of ulcerative colitis.

### Example 53

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treatment continued accordingly to prevent the occurrence of ulcerative colitis.

### Example 54

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 25-50 mg/kg of alkaline protease every week for 6 weeks. The human patient is monitored and treatment continued accordingly to prevent the occurrence of ulcerative colitis.

### Example 55

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising 1750-3500 mg of alkaline protease every day for 7-10 days. The human patient is monitored and treatment continued accordingly to prevent the occurrence of ulcerative colitis.

### Example 56

### Prophylaxis of Crohn's Disease

A human patient diagnosed with risk factors for Crohn's disease can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treatment continued accordingly to prevent the occurrence of ulcerative colitis.

### Example 57

### Prophylaxis of Crohn's Disease

A human patient diagnosed with risk factors for Crohn's disease can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treatment continued accordingly to prevent the occurrence of ulcerative colitis.

### Example 58

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days. The human patient is monitored and treated according to the lessening of the symptoms and/or the development of side effects.

### Example 59

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 25-50 mg/kg of alkaline protease every day for 7-10 days, wherein said pharmaceutical composition has a specific activity where it cleaves Val-leu-lys-pNA at least about 100 mOD/min/μg protein. The human patient is monitored and treated according to the lessening of the symptoms and/or the development of side effects.

### Example 60

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 25-50 mg/kg of alkaline protease every week for 6 weeks. The human patient is monitored and treated according to the lessening of the symptoms and/or the development of side effects.

### Example 61

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising 1750-3500 mg of alkaline protease every day for 7-10 days. The human patient is monitored and treated according to the lessening of the symptoms and/or the development of side effects.

### Example 62

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. To begin treatment, the human patient is injected with of a pharmaceutical composition comprising 10 μg of alkaline protease at 0, 12, 24, and then 36 hours. The injections of a pharmaceutical composition comprising 10 μg alkaline protease are continued once a day for 7-10 days. The human patient is monitored and treated according to the lessening of the symptoms and/or the development of side effects.

### Example 63

### Prophylaxis of Recurrence or a Relapse of Crohn's Disease

A human patient previously diagnosed with Crohn's disease who is not instantly experiencing any symptoms can be treated by administering a pharmaceutical composition comprising alkaline protease. The human patient is orally administered a pharmaceutical composition comprising A. oryzae alkaline protease every day for 7-10 days, where each dose of the pharmaceutical composition has enough alkaline protease to cleave 1.2-2.5 mmol of peptide bonds in Salmon proteamine under the assay conditions of Example 5. The human patient is monitored and treated according to the lessening of the symptoms and/or the development of side effects.

Although the invention has been described in some detail by way of illustration and example for purposes of clarity of understanding, it will be obvious that certain changes and modifications may be practiced within the scope of the appended claims. Modifications of the above-described modes for carrying out the invention that are obvious to persons of skill in medicine, pharmacology, microbiology and/or related fields are intended to be within the scope of the following claims.

All publications and patent applications mentioned in this specification are indicative of the level of skill of those skilled in the art to which this invention pertains. All such publications and patent applications are herein incorporated by reference to the same extent as if each individual publication or patent application was specifically and individually indicated to be incorporated by reference.

1. A method of treating an inflammatory condition involving TNF-α in a mammal comprising administering to said mammal a composition comprising an effective amount of an isolated alkaline protease in an amount effective to inactivate TNF-α.

2. The method of claim 1, wherein the inflammatory condition is selected from the group consisting of ulcerative colitis, inflammatory bowel disease, asthma, Parkinson's disease (PD), cardiovascular disease, Crohn's disease (CD), multiple sclerosis (MS), irritable bowel syndrome (IBD), Alzheimer's disease (AD), infection, soft tissue injury, encephalitis, Graves' disease, myasthenia gravis, rheumatoid arthritis (RA), scleroderma, acute rheumatic fever, Kawasaki disease (KD), cachexia syndrome, acute pancreatitis, and chronic heart failure (CHF).

3. The method of claim 1, wherein said composition does not include a 26 kDa protease or deuterolysin.

4. The method of claim 1, wherein said composition consists essentially of an isolated alkaline protease in an amount effective to inactive TNF-α.

5. The method of claim 1, wherein said composition has a maximum proteolytic activity at about pH=8.0.

6. The method of claim 1, wherein said isolated alkaline protease is an Aspergillus oryzae alkaline protease.

7. The method of claim 6, wherein said isolated Aspergillus oryzae alkaline protease comprises SEQ ID NO: 2.

8. The method of claim 6, wherein said isolated Aspergillus oryzae alkaline protease is recombinantly produced.

9. The method of claim 1, wherein said composition is administered orally.

10. The method of claim 1, wherein said composition comprises a pharmaceutically acceptable carrier, excipient, diluent, or solution.

11. The method of claim 1, wherein said composition is a food supplement, a nutritional supplement, or a food product.

12. Use of a composition for medical therapy comprising an isolated alkaline protease which inactivates TNF-α, wherein said protease comprises SEQ ID NO: 2.

13. The use of a composition according to claim 12, wherein said composition comprises a pharmaceutically acceptable carrier, excipient, diluent, or solution.

14. The use of a composition according to claim 12, wherein said composition is a food supplement, a nutritional supplement, or a food product.

15. The use of a composition according to claim 12, wherein said composition has an optimal activity of about pH=7.0-10.0.

16. The use of a composition according to claim 12, wherein the medical therapy is for an inflammatory condition.

17. The use of a composition according to claim 16, wherein the inflammatory condition involved TNF-α.

18. The use of a composition according to claim 16, wherein the inflammatory condition is selected from the group consisting of ulcerative colitis, inflammatory bowel disease, asthma, cardiovascular disease, Crohn's disease, Parkinson's Disease (PD), multiple sclerosis (MS), irritable bowel syndrome (IBS), Alzheimer's disease (AD), infection, soft tissue injury, encephalitis, Graves' disease, myasthenia gravis, rheumatoid arthritis (RA), sclerodema, acute rheumatic fever, Kawasaki disease (KD), cachexia syndrome, acute pancreatitis, and chronic heart failure (CHF).
